SEQUENCE LISTING (22 sequences)

SEQ ID NO: 1 - DNA, 29 bp, Artificial Sequence
  Synthesized forward primer for COL5A1 (A/T) rs1134170
cttgtggtgctatctatctgttttaaggt

SEQ ID NO: 2 - DNA, 24 bp, Artificial Sequence
  Synthesized reverse primer for COL5A1 (A/T) rs1134170
ggataaagaaagcagggagaacga

SEQ ID NO: 3 - DNA, 25 bp, Artificial Sequence
  Synthesised forward primer for COL5A1 (-/AGGG) rs71746744
gccccttttcagacagtttttgatt

SEQ ID NO: 4 - DNA, 28 bp, Artificial Sequence
  Synthesised reverse primer for COL5A1 (-/AGGG) rs71746744
caaacctgtgttttagtcttaagtgcat

SEQ ID NO: 5 - DNA, 20 bp, Artificial Sequence
  Synthesised forward primer for IL-1Beta (T>C) (rs1143627)
gcctgaaccctgcataccgt

SEQ ID NO: 6 - DNA, 20 bp, Artificial Sequence
  Synthesised reverse primer for IL-1Beta (T>C) (rs1143627)
gccaatagccctccctgtct

SEQ ID NO: 7 - DNA, 34 bp, Artificial Sequence
  Synthesised forward primer for IL-6 (G/C) (rs1800795)
ttttctctttgtaaaacttcgtgcataactactt

SEQ ID NO: 8 - DNA, 26 bp, Artificial Sequence
  Synthesised reverse primer for IL-6 (G/C) (rs1800795)
tggggctgattggaaaccttattaag

SEQ ID NO: 9 - DNA, 23 bp, Artificial Sequence
  Synthesised forward primer for COL5A1 (ATCT/-) rs16399
ttctctcttgtggctctcttgtg

SEQ ID NO: 10 - DNA, 17 bp, Artificial Sequence
  Synthesised reverse primer for COL5A1 (ATCT/-) rs16399
cccagtgcgccttcaag

SEQ ID NO: 11 - DNA, 29 bp, Artificial Sequence
  Synthesised forward primer for CASP8 (CTTACT/del) (rs3834129)
ttgattctttcagactttttcctaggctt

SEQ ID NO: 12 - DNA, 25 bp, Artificial Sequence
  Synthesised reverse primer for CASP8 (CTTACT/del) (rs3834129)
ggaaggcactgagacgttaagtaac

SEQ ID NO: 13 - DNA, 21 bp, Artificial Sequence
  Synthesised forward primer for CASP8 (G/C) D302H (rs1045485)
accacgacctttgaagagctt

SEQ ID NO: 14 - DNA, 30 bp, Artificial Sequence
  Synthesised reverse primer for CASP8 (G/C) D302H (rs1045485)
tccatgagttggtagattttcaaaatctca

SEQ ID NO: 15 - DNA, 20 bp, Artificial Sequence
  Synthesised forward primer for IL-1Beta  (C/T) (rs16944)
tggcattgatctggttcatc

SEQ ID NO: 16 - DNA, 19 bp, Artificial Sequence
  Synthesised reverse primer for IL-1Beta  (C/T) (rs16944)
gtttaggaatcttcccact

SEQ ID NO: 17 - DNA, 23 bp, Artificial Sequence
  Synthesised forward primer for GDF5 (T/C) (rs143383)
tcagtcagttgtgcaggagaaag

SEQ ID NO: 18 - DNA, 26 bp, Artificial Sequence
  Synthesised reverse primer for GDF5 (T/C) (rs143383)
gcagctgaaaataactcgttcttgaa

SEQ ID NO: 19 - DNA, 25 bp, Artificial Sequence
  Synthesised sequence of Has-miR-608 with a C at the 22nd position
aggggtggtgttgggacagctccgt

SEQ ID NO: 20 - DNA, 25 bp, Artificial Sequence
  Synthesised sequence of Has-miR-608 with a G at the 22nd position
aggggtggtgttgggacagctgcgt

SEQ ID NO: 21 - DNA, 100 bp, Homo sapiens
  gene (1)..(100)
  gene (1)..(100): Sequence of the MiR608 gene (ENSE00001499827)
gggccaaggtgggccaggggtggtgttgggacagctccgtttaaaaaggcatctccaagagcttccatcaaaggctgcctcttggtgcagcacaggtaga

SEQ ID NO: 22 - DNA, 52 bp, Homo sapiens
  variation (1)..(52): Sequence of the rs4919510 polymorphism
gggccaggggtggtgttgggacagctgcgtttaaaaaggcatctccaagagc

(19) United States
(12) Patent Application Publication
(10) Pub. No.: US 2015/0057171 A1
(43) Pub. Date: Feb. 26, 2015

(54) OLIGONUCLEOTIDES AND METHODS FOR DETERMINING A PREDISPOSITION TO SOFT TISSUE INJURIES

(71) Applicants: SOUTH AFRICAN MEDICAL RESEARCH COUNCIL, Cape Town (ZA); UNIVERSITY OF CAPE TOWN, Cape Town (ZA)

(72) Inventors: Malcolm Robert Collins, Cape Town (ZA); Alison Vicotria September, Cape Town (ZA)

(21) Appl. No.: 14/370,589

(22) PCT Filed: Jan. 4, 2013
(86) PCT No.: PCT/IB2013/050083

§ 371 Date: Jul. 3, 2014

(30) Foreign Application Priority Data
Jan. 4, 2012 (ZA) 2012/00047

(51) Int. Cl. C12Q 1/68

(52) U.S. Cl. CPC C12Q 1/6883; C12Q 2600/156; C12Q 2600/118; C12Q 2600/172; C12Q 2600/178
USPC 506/9; 435/6.11

(57) ABSTRACT

A method of determining in a subject a predisposition to, or increased risk for, developing a tendon, ligament, or other soft tissue injury or pathology, the method comprising the step of screening the subject for the presence of at least one polymorphism in at least one gene selected from the group comprising: a) the collagen V gene COL5A1; wherein the COL5A1 gene is rs71746744, rs16399 and/or rs1134170 within the 3′-untranslated region (UTR) of the alpha 1 chain of the COL5A1 gene; b) the MIR608 gene which encodes a miRNA which binds to a recognition sequence within the 3′-UTR of the collagen V gene COL5A1; and c) the CASP8 gene; wherein the presence of the polymorphism is indicative of a predisposition to, or increased risk for, developing a musculoskeletal soft tissue injury in the subject.

### FIELD OF THE INVENTION

THIS INVENTION relates to methods of determining in a subject a predisposition to, or increased risk for, developing a tendon, ligament, or other soft tissue injury or pathology. The invention also relates to molecular markers; isolated nucleic acid molecules; primers and oligonucleotide sets; and detection reagents capable of detecting one or more single nucleic acid polymorphisms; for use therein.

### BACKGROUND TO THE INVENTION

Tendon and ligament pathologies as well as exercise associated muscle cramping (EAMC) can affect subjects participating in a range of sporting pursuits, as well as occurring in the less physically active (Kader et al. Br J Sports Med 2002; 36:239-49; Young et al. Foot Ankle Clin 2005; 10: 371-382). These pathologies affect soft tissues, such as skeletal muscles, tendons and ligaments, and their surrounding structures (Puddu et al. Am J Sports Med 1976; 4:145-50), and include, for example, Achilles tendinopathy (AT), acute spontaneous rupture, and injury to the anterior cruciate ligament (ACL).

Achilles tendinopathy (AT) is a degenerative condition involving inflammation of the Achilles tendon and is often caused by overuse or mechanical overload of the Achilles tendon. Acute spontaneous rupture also commonly affects the Achilles tendon, particularly in the middle-aged, male athlete. Injury to the anterior cruciate ligament (ACL) is one of the more severe sporting injuries, the risk of which is increased by movements involving a sudden deceleration or change in direction.

A number of intrinsic and extrinsic factors have been implicated in raising the risk of these pathologies (Jarvinen et al. Foot Ankle Clin 2005; 10: 255-266). Intrinsic factors include genetic variability in several genes that are known to be associated with increased risk of these pathologies. These genes include, for example, the α chain of type V collagen (COL5A1), tenascin C (TNC), enzymes that breakdown the matrix such as matrix metalloproteinases (MMP-3), and inflammatory process genes such as the inflammatory cytokine, growth differentiating factor, and IL-1β, IL-1RN and IL-6 (September et al., Br J Sports Med 2011; 45:1040-1047). Polymorphisms in some of these genes have been found to be associated with exercise related phenotypes (Collins and Posthumus, Exercise and Sport Sciences Reviews, 2011 39(4), 191-198) including AT (Mokone et al. Scand J Med Sci Sports 2006; 16:19-26; September et al. Br J Sports Med 2009; 43:357-365; Jarvinen et al. J Cell Sci 2003; 116(Pt 5):857-866; Jarvinen et al. J Cell Sci 1999; 112 (Pt 18):3157-3166); anterior curaciate ligament ruptures (ACL) (Posthumus et al. Am J Sports Med 2009; 37(11), 2234-2240). More recently polymorphisms with COL5A1 were associated with ROM (Collins et al. Scand J Med Sci Sports 2009, 19(6), 803-810; Brown et al. Scand J Med Sci Sports, 2011 21(6), e266-72.) and athletic performance (Posthumus et al. Med Sci Sports Exerc 2011, 43(4), 584-589; Brown et al., IJSPP 2011 in press).

Extrinsic factors include, for example, repetitive loading which may impede repair of damages tendons. For instance, tenocytes are required to maintain homeostasis of the extracellular matrix (ECM) by regulating the balance between ECM synthesis and degradation (Clancy American Orthopedic Society for Sports Medicine: Park Ridge, II. 1989), During the normal tendon healing process damaged tenocytes are removed by cytokine-mediated apoptosis. Repetitive loading may, however, change the extracellular matrix (ECM) composition and result in excessive tenocyte apoptosis (Yuan et al. J Orthop Res 2002; 20:1372-1379; Egerbacher et al. Clin Orthop Relat Res 2008; 466:1562-1568). Excessive tenocyte apoptosis which has been observed in tendinopathy (Scott et al. Br J Sports Med 2005; 39:e25) may compromise the ability of the tendon to regulate repair processes.

There is a need for improved methods of determining in a subject a predisposition to, or increased risk for, developing a tendon, ligament, or other soft tissue injury or pathology.

### BRIEF SUMMARY OF THE INVENTION

According to one aspect of the invention, there is provided a method of determining in a subject a predisposition to, or increased risk for, developing a tendon, ligament, or other soft tissue injury or pathology, the method comprising the step of screening the subject for the presence of at least one polymorphism in at least one gene selected from the group comprising any one or more of:
a) the collagen V gene COL5A1; wherein the COL5A1 gene is rs71746744, rs16399 and/or rs1134170 within the 3′-untranslated region (UTR) of the alpha 1 chain of the COL5A1 gene;
b) the MIR608 gene which encodes a miRNA which binds to a recognition sequence within the 3′-UTR of the collagen V gene COL5A1; and
c) the CASP8 gene;
   which polymorphism is a polymorphism which results in a modified, augmented, or mitigated interaction with one or more other genes selected from the group, when compared to a wild-type interaction and wherein the presence of the polymorphism is indicative of a predisposition to, or increased risk for, developing a musculoskeletal soft tissue injury in the subject.

The tendon, ligament, or other soft tissue injury or pathology, may be selected from the group including tendon injuries, ligament injuries, EAMC, ROM, and endurance running performance.

According to another aspect of the invention, there is provided a method of determining in a subject a predisposition to, or increased risk for, developing a tendon, ligament, or other soft tissue injury or pathology, the method comprising the step of screening the subject for the presence of at least one polymorphism within a collagen V gene COL5A1, and at least one polymorphism in at least one gene selected from the comprising:
a) the GDF5 gene;
b) the IL6 gene; and
c) the IL1B gene;
d) MIR608 gene; and
e) the CASP8 gene;
   which polymorphism is a polymorphism which results in a modified, augmented, or mitigated interaction with one or more polymorphisms described herein, when compared to a wild-type interaction and wherein the presence of the polymorphism is indicative of a predisposition to, or increased risk for, developing a musculoskeletal soft tissue injury in the subject.

The method may further include the step of screening the subject for gender.

The polymorphism of the COL5A1 gene may be rs71746744, rs16399 and/or rs1134170 within the 3′-untranslated region (UTR) of the alpha 1 chain of the COL5A1 gene. The polymorphism of the MIR608 gene may be rs4919510. The polymorphism of the CASP8 gene may be rs1045485 and rs3834129.

More particularly, the polymorphism of the COL5A1 gene may be rs71746744 (-/AGGG), rs16399 (ATCT/-) and/or rs1134170 (A/T) within the 3′-untranslated region (UTR) of the alpha 1 chain of the COL5A1 gene. The polymorphism of the MIR608 gene may be rs4919510 (C/G). The polymorphism of the CASP8 gene may be rs1045485 (G/C, D302H) and rs3834129 (CTTACT/del).

More specifically, the method may include the step of detecting or screening for the presence of a polymorphism in the COL5A1 gene which has modified, augmented, or mitigated interaction with a MIR608 polymorphism product or a CASP8 gene product, when compared to a wild-type interaction. More particularly, the COL5A1 gene polymorphism may be a polymorphism which has a modified, augmented, or mitigated interaction with the rs4919510 (C/G) MIR608 polymorphism, and/or the rs1045485 (G/C, D302H) CASP8 polymorphism; and/or the rs3834129 (CTTACT/del) CASP8 polymorphism, and/or any other linked polymorphism, and the product encoded thereby.

The polymorphism of the COL5A1 gene may be rs71746744, rs16399 and/or rs1134170 within the 3′-untranslated region (UTR) of the alpha 1 chain of the COL5A1 gene. The polymorphism of the GDF5 gene may be rs143383. The polymorphism of the CASP8 gene may be rs1045485 and/or rs3834129. The polymorphism of the IL6 gene may be rs1800795. The polymorphism of the IL1B gene may be rs1143627 and/or rs16944. The polymorphism of the MIR608 gene may be rs4919510.

More particularly, the polymorphism of the COL5A1 gene may be rs71746744 (-/AGGG), rs16399 (ATCT/-) and/or rs1134170 (A/T) within the 3′-untranslated region (UTR) of the alpha 1 chain of the COL5A1 gene. The polymorphism of the GDF5 gene may be rs143383 (T/C). The polymorphism of the CASP8 gene may be rs1045485 (G/C, D302H) and/or rs3834129 (CTTACT/del). The polymorphism of the IL6 gene may be rs1800795 (G/C). The polymorphism of the IL1B gene may be rs1143627 (T/C) and/or rs16944 (C/T). The polymorphism of the MIR608 gene may be rs4919510 (C/G).

According to another aspect of the invention, there is provided a molecular marker for use in diagnosing a predisposition to, or increased risk for, developing tendon, ligament, or other soft tissue pathology or injury in a subject, the molecular marker comprising any one or more of:
a) at least one isolated nucleic acid fragment derived from a COL5A1 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof, wherein the COL5A1 gene has one or more of the following polymorphisms: rs71746744, rs16399 and/or rs1134170 in the alpha 1 chain of the COL5A1 gene;
b) at least one isolated nucleic acid fragment derived from a MIR608 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof;
c) at least one isolated nucleic acid fragment derived from a CASP8 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof;
d) at least one isolated nucleic acid fragment derived from a GDF5 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof;
e) at least one isolated nucleic acid fragment derived from a IL6 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof; and
f) at least one isolated nucleic acid fragment derived from a IL1B gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof.

The tendon, ligament, or other soft tissue pathology or injury may be EAMC.

The molecular marker may be DNA-based, RNA-based, or other combinations of nucleic acids or modified bases.

The molecular marker may comprise an isolated nucleic acid fragment that is a part of, or a fragment derived from, the group comprising a COL5A1 gene, a MIR608 gene, a CASP8 gene, a GDF5 gene, a IL6 gene, and a IL1B gene, the fragment being between 10 and 40, preferably between 15 and 35, more preferably between 20 and 30 nucleic acids in length, and which hybridizes under stringent hybridization conditions to at least a portion of the COL5A1 gene, the MIR608 gene, the CASP8 gene, the GDF5 gene, the IL6 gene, or the IL1B gene. This may include sequences complementary to the marker, and sequences having substitutions, deletions or insertions, sequences which can hybridize under strict hybridization conditions thereto, and functional discriminatory truncations thereof.

In one embodiment, the molecular marker is a polymorphic sequence variant or a polymorphism. The polymorphism may be any one or more of the polymorphisms selected from the group comprising rs71746744, rs16399 and rs1134170 within the 3′-untranslated region (UTR) of the alpha 1 chain of the COL5A1 gene; rs4919510 of the MIR608 gene; rs1045485 and rs3834129 of the CASP8 gene; rs143383 of the GDF5 gene; rs1800795 of the IL6 gene; and rs1143627, rs16944 of the IL1B gene; together with any other polymorphism closely linked (i.e. which is in high linkage disequilibrium) with any of the specific polymorphisms listed above.

More particularly, the polymorphisms may be selected from the group comprising:
a) rs71746744 (-/AGGG), rs16399 (ATCT/-) and rs1134170 (A/T) within the 3′-untranslated region (UTR) of the alpha 1 chain of the COL5A1 gene;
b) rs4919510 (C/G) of the MIR608 gene;
c) rs1045485 (G/C, D302H) and rs3834129 (CTTACT/del) of the CASP8 gene;
d) rs143383 (T/C) of the GDF5 gene;
e) rs1800795 (G/C) of the IL6 gene; and
f) rs1143627 (T/C) and rs16944 (C/T) of the IL1B gene.

More particularly, the molecular marker may be, or may be detectable using, any one or more isolated oligonucleotides selected from the group comprising: SEQ. ID. NO. 1 to SEQ. ID. NO. 18; sequences complementary thereto, sequences which can hybridize under stringent hybridization conditions thereto, and functional discriminatory truncations thereof.

Accordingly, the invention extends to a primer or oligonucleotide sets for use in detecting or diagnosing a predisposition to, or increased risk for, developing tendon, ligament, or other soft tissue pathologies or injuries in a subject, the primer or oligonucleotide sets comprising isolated nucleic acid sequences selected from the group comprising: Set 1: SEQ. ID. NO. 1 and SEQ. ID. NO. 2; Set 2: SEQ. ID. NO. 3 and SEQ. ID. NO. 4; Set 3: SEQ. ID. NO. 5 and SEQ. ID. NO. 6; Set 4: SEQ. ID. NO. 7 and SEQ. ID. NO. 8; Set 5: SEQ. ID. NO. 9 and SEQ. ID. NO. 10; Set 6: SEQ. ID. NO. 11 and SEQ. ID. NO. 12; Set 7: SEQ. ID. NO. 13 and SEQ. ID. NO. 14; Set 8: SEQ. ID. NO. 15 and SEQ. ID. NO. 16; Set 9: SEQ. ID. NO. 17 and SEQ. ID. NO. 18; sequences complementary thereto, sequences which can hybridize under stringent hybridization conditions thereto, and functional discriminatory truncations thereof.

According to a further aspect of the invention, there is provided an isolated nucleic acid molecule for detecting at least one SNP provided hereinbefore, wherein the nucleic acid molecule comprises less than 40, less than 30, less than 20, or even preferably less than 10 contiguous nucleotides selected from the group comprising SEQ ID NOS 1 to 18, and fragments, complementary sequences, sequences which can hybridize under stringent hybridization conditions thereto, and functional discriminatory truncations thereof.

The invention extends also to a detection reagent capable of detecting one or more single nucleic acid polymorphisms selected from the group comprising the polymorphisms listed hereinbefore, fragments thereof, sequences complementary thereto, sequences which can hybridize under stringent hybridization conditions thereto, and functional discriminatory truncations thereof.

According to another aspect of the invention, there is provided a diagnostic assay comprising any one or more of the markers described hereinbefore, fragments thereof, sequences complementary thereto, sequences which can hybridize under stringent hybridization conditions thereto, and functional discriminatory truncations thereof.

According to yet another aspect of the invention, there is provided a method of determining a predisposition for, or increased risk of, developing a tendon, ligament and/or soft tissue pathology or injury in a subject, the method comprising the steps of screening a subject for a polymorphism in one or more of the following genes:
a) the collagen V gene COL5A1; wherein the COL5A1 gene is rs71746744, rs16399 and/or rs1134170 within the 3′-untranslated region (UTR) of the alpha 1 chain of the COL5A1 gene;
b) the MIR608 gene which encodes a miRNA which binds to a recognition sequence within the 3′-UTR of COL5A1; and
c) the CASP8 gene.

According to another aspect of the invention, there is provided a method of determining a predisposition for, or increased risk of, developing a tendon, ligament and/or soft tissue pathology or injury in a subject, the method comprising the step of screening the subject for the presence of at least one polymorphism in the collagen V gene, COL5A1, and at least one polymorphism in at least one gene selected from the group comprising:
a) the GDF5 gene;
b) the IL6 gene; and
c) the IL1B gene.

According to a further aspect of the invention, there is provided a method of diagnosing a predisposition to, or increased risk for, developing a tendon, ligament, or other soft tissue injury or pathology, the method comprising the steps of:
a) obtaining a biological sample from a subject, the biological sample comprising nucleic acid;
b) detecting the presence or absence in the biological sample of at least one polymorphism in at least one gene selected from the group comprising any one or more of:
   i) the collagen V gene COL5A1; wherein the COL5A1 gene is rs71746744, rs16399 and/or rs1134170 within the 3′-untranslated region (UTR) of the alpha 1 chain of the COL5A1 gene;
   ii) the MIR608 gene which encodes a miRNA which binds to a recognition sequence within the 3′-UTR of the collagen V gene COL5A1; and
   iii) the CASP8 gene;
   wherein the polymorphism is a polymorphism which results in a modified, augmented, or mitigated interaction with one or more other genes selected from the group, when compared to a wild-type interaction and wherein the presence of the polymorphism is indicative of a predisposition to, or increased risk for, developing a musculoskeletal soft tissue injury in the subject.

The tendon, ligament, or other soft tissue injury or pathology, may be selected from the group including tendon injuries, ligament injuries, EAMC, ROM, and endurance running performance.

According to another aspect of the invention, there is provided a method of diagnosing a predisposition to, or increased risk for, developing a tendon, ligament, or other soft tissue injury or pathology, the method comprising the steps of
a) obtaining a biological sample from a subject, the biological sample comprising nucleic acid;
b) detecting the presence or absence in the biological sample of at least one polymorphism within a collagen V gene COL5A1, and at least at least one polymorphism in at least one gene selected from the group comprising one or more of the following genes:
   i) the GDF5 gene;
   ii) the IL6 gene; and
   iii) the IL1B gene;
   iv) MIR608 gene; and
   v) the CASP8 gene;
      wherein the polymorphism is a polymorphism which results in a modified, augmented, or mitigated interaction with one or more polymorphisms described herein, when compared to a wild-type interaction and wherein the presence of the polymorphism is indicative of a predisposition to, or increased risk for, developing a musculoskeletal soft tissue injury in the subject.

The polymorphism may be any one of more of the polymorphisms listed hereinbefore, polymorphisms in high linkage disequilibrium with the listed polymorphisms, or a polymorphism detectable using any one or more of the sequences listed hereinbefore, fragments thereof, sequences complementary thereto, sequences which can hybridize under stringent hybridization conditions thereto, and functional discriminatory truncations thereof.

The method may further include the step of screening the subject for gender.

The method may include the additional steps of:
a) providing a tissue sample from a subject;
b) extracting nucleic acid from the sample;
c) amplifying selected regions of the nucleic acid using any one or more of the molecular markers selected from the group comprising: SEQ. ID. NOs 1 to 6, thereby to obtain amplified nucleic acid fragments; and
d) screening the amplified nucleic acid fragments for the presence of the polymorphisms listed hereinbefore.

According to another aspect of the invention, there is provided use of a molecular marker of the invention in diagnosing a predisposition to a soft tissue pathology in a subject.

According to a still further aspect of the invention, there is provided a kit for use in diagnosing a predisposition to a soft tissue pathology in a subject, the kit comprising:
a) any one or more of the molecular markers selected from the group comprising: SEQ. ID. NOs 1 to 18; and
b) suitable reaction media.

The kit may further include any one or more of reagents, such as buffers, DNases, RNAses, polymerases, instructions, and the like.

The molecular markers may be any one or more markers selected from the markers listed hereinbefore.

The soft tissue may be a connective tissue injury, and may include tendon and/or ligament injuries such as, for example, Achilles tendon, knee ligament and ankle ligament pathologies. The sample may comprise an animal tissue or blood sample, such as a human tissue or blood sample.

Further features of the invention will now be described with reference to the following non-limiting examples and figures.

### DETAILED DESCRIPTION OF THE INVENTION

In the drawings:

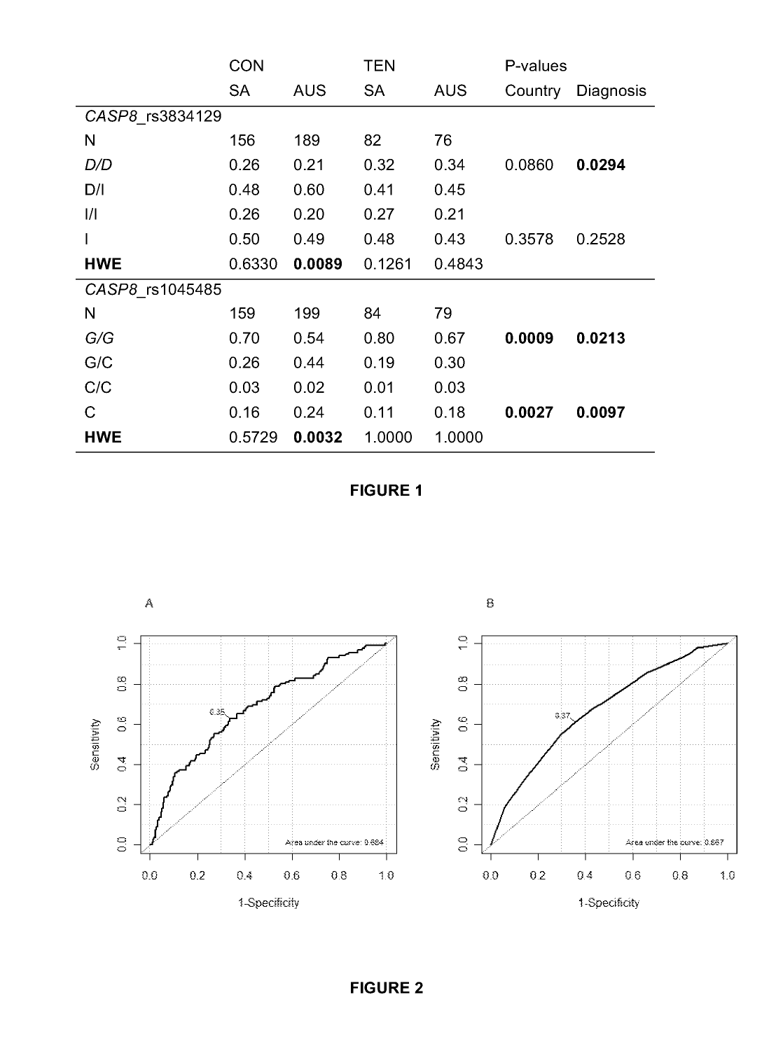

FIG. 1 shows a table setting out genotype frequency distributions and minor allele frequencies of CASP8_rs3834129, CASP8_rs1045485, NOS3_rs1799983 and NOS2_rs2779249 polymorphisms in control (CON) and Achilles tendinopathy (TEN) groups of South Africa (SA) and Australia (AUS). P-values are for the difference between countries and between diagnostic groups respectively, adjusted for each other, age, gender and whether or not a person was investigated in his/her country of birth. HWE are exact p-values from tests of Hardy-Weinberg equilibrium. The genotype p-value is from a 2 degree of freedom test, with genotypes as categories. The allelic p-value is from additive allelic model on logit scale. N is number of samples genotyped.

FIG. 2 shows graphs demonstrating the receiver operating characteristic (ROC) curve of the apoptosis cascade profile (bold curve) to determine the true positive (sensitivity) versus true negative (specificity) rate for various cut-offs in determining risk of Achilles tendinopathy; the straight line indicates where sensitivity=1-specificity and AUC=0.5. The optimal cut-off which yields the maximum sensitivity plus specificity is indicated on the graph with an arrow. (A) The logistic regression model containing the confounders sex, age, country and born-here and the genotype data from rs384129, rs1045485, rs1799983, rs2779249) to predict AT risk; AUC=0.684; sensitivity=62.9% and specificity=66.2%. CON=159; TEN=93. (B) The optimal model containing sex and genotype data from rs384129 and rs1045485. AUC=0.667; sensitivity=60.9% and specificity=64.3%. CON=336; TEN=151.

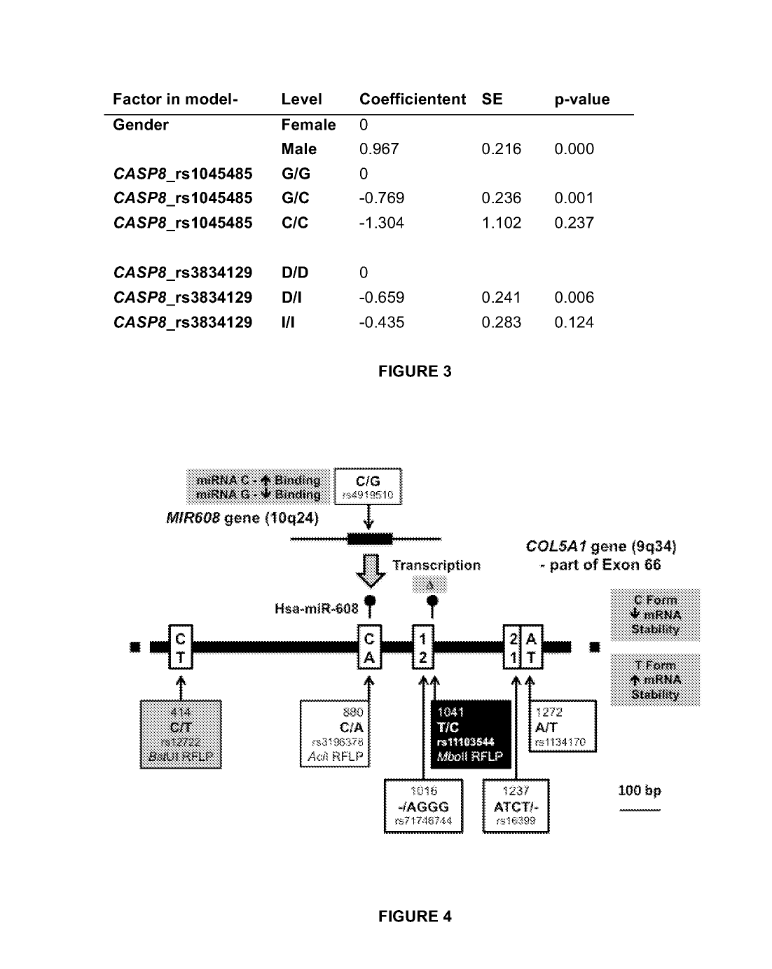

FIG. 3 shows a table summarising the optimal logistic regression model used for ROC analysis. The coefficients are used to calculate points on the ROC curve. P-values are from joint model, so adjusted for each other, all assessing the effect of specific factor level compared to reference level—the absent one (female; G/G and D/D respectively). Examples of calculating the estimates for the ROC curve in FIG. 2.B: prediction for female, G/G (rs1045485) and D/D (rs3834129)=−0.735; prediction for male, G/G (rs1045485) and D/D (rs3834129)=−0.735+0.967; prediction for male, G/C (rs1045485) and D/D (rs3834129)=−0.735+0.967-0.769. If the model is a good predictor, large values will indicate TEN cases and small values CON cases.

FIG. 4 shows a schematic representation of the region (from SNP rs12722 to rs1134170) within the 3′-untranslated region (UTR) of the human COL5A1 gene on chromosome 9q34 associated with several exercise-associated phenotypes and the MIR608 gene on chromosome 10q24. Five of the seven polymorphic sites which distinguish the C and T functional forms of the COL5A1 3′-UTR are annotated in the white or grey boxes. The downstream and upstream single nucleotide polymorphisms (SNPs) rs13946 (DpnII RFLP, C/T) and rs3128575 (C/T), respectively, are not shown. SNP rs12722 (BstUI RFLP) previously associated with several exercise-related phenotypes is indicated in the grey box. Although not associated with the C and T functional forms of the COL5A1 3′-UTR, SNP rs11103544 (MboII RFLP) is within the second putative miRNA binding site and is therefore also annotated within a black box. The single SNP within the MIR608 gene is also annotated. The accession numbers and/or RFLP associated with the polymorphism are indicated together with the nucleotide changes. The nucleotide positions of the polymorphisms within the 3′-UTR are for the wild-type sequence (C functional form). The two miRNA binding sites are indicated by a black solid circle and line. The location of a previously described 57 bp region ( ) containing the second miRNA binding site, rs71746744 and rs11103544 is also indicated.

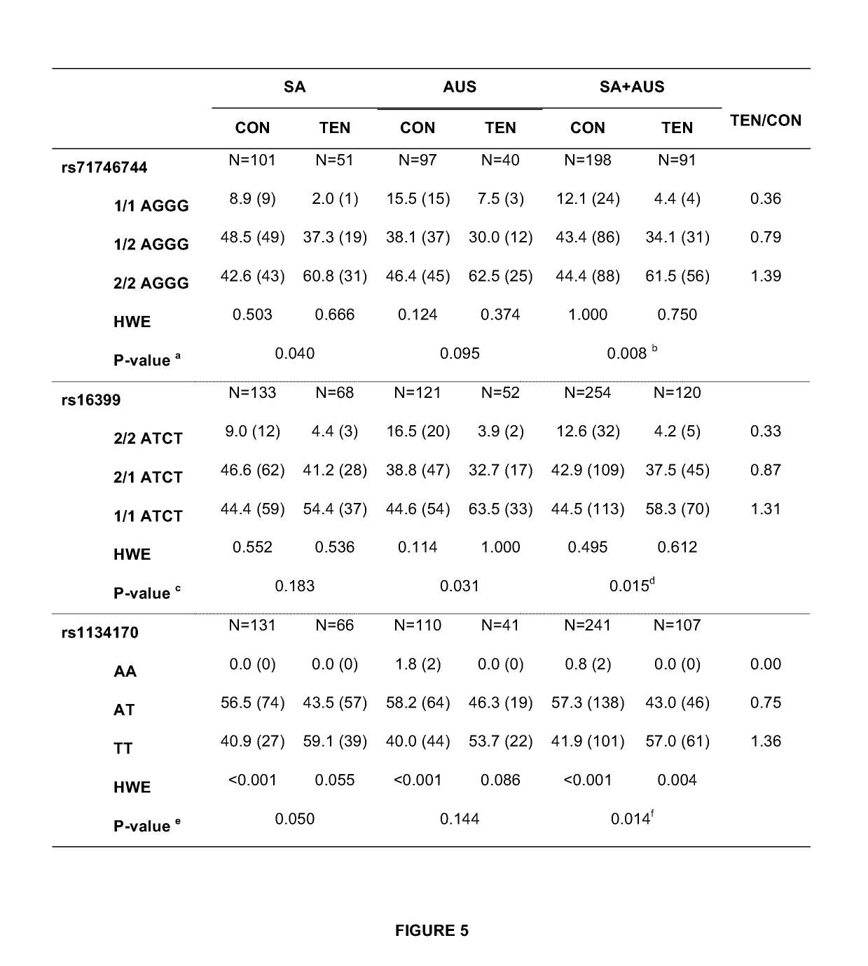

FIG. 5 shows a table summarising genotype frequency distributions the COL5A1 3′-untranslated region (UTR) polymorphisms, rs71746744 (-/AGGG), rs16399 (ATCT/-) and rs1134170 (A/T), in control (CON) and chronic Achilles tendinopathy (TEN) groups of South African (SA) and Australian (AUS) cohorts, as well as the combined SA and AUS (SA+AUS) cohorts. Genotypes are expressed as percentages with numbers (N) in parenthesis. HWE are exact p-values from tests of Hardy-Weinberg equilibrium. ^{a}2/2 AGGG genotype vs 1 AGGG allele. ^{b}odd ratio=2.0, 95% confidence interval=1.2 to 3.3. ^{c}1/1 ATCT genotype vs 1 ATCT allele. ^{d}odd ratio=1.7, 95% confidence interval=1.1 to 2.7. ^{e}TT genotype vs A allele (AT and TT genotypes). ^{f}odd ratio=1.8, 95% confidence interval=1.1 to 2.9

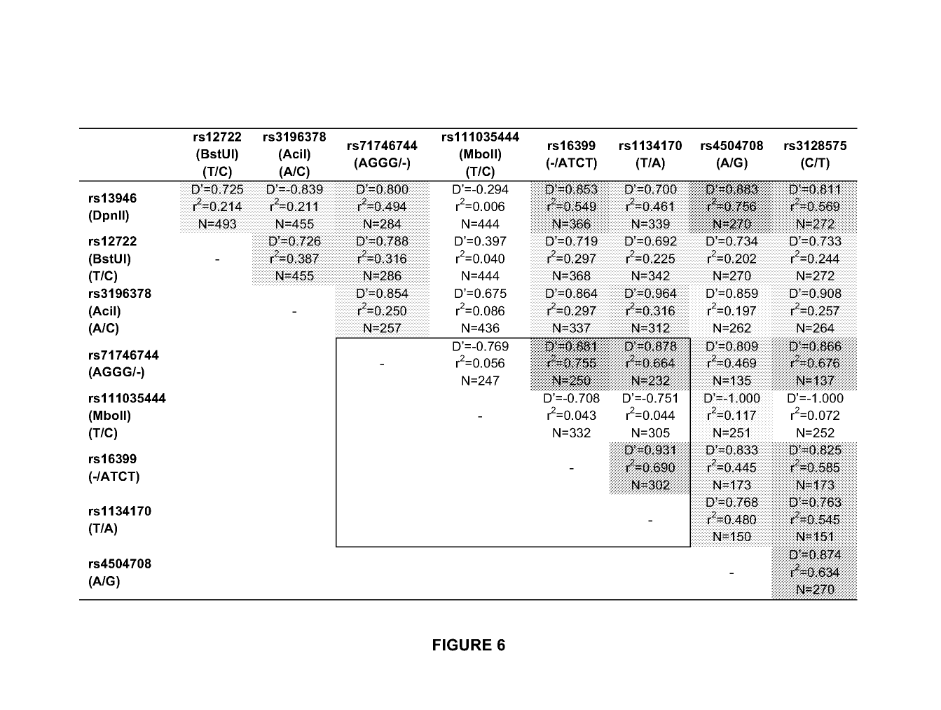

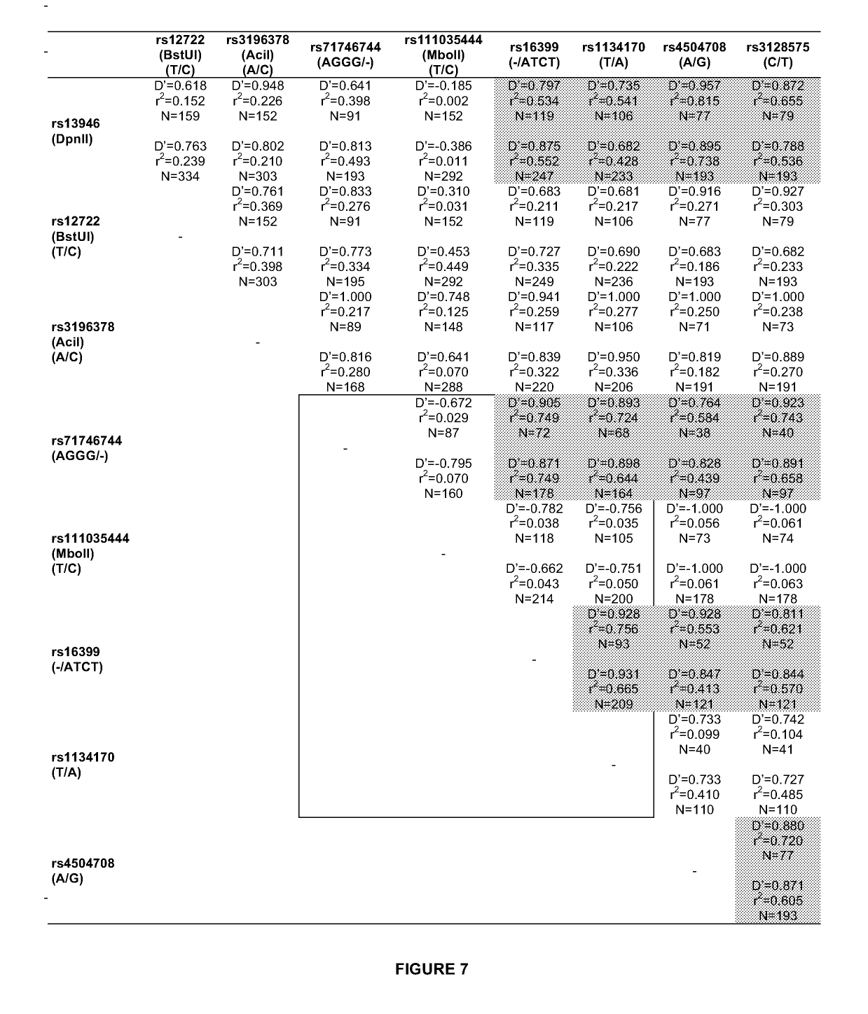

FIGS. 6 and 7 show tables summarizing the Linkage Disequilibrium (LD) between eight of the common variants within the COL5A1-3′ UTR described herein.

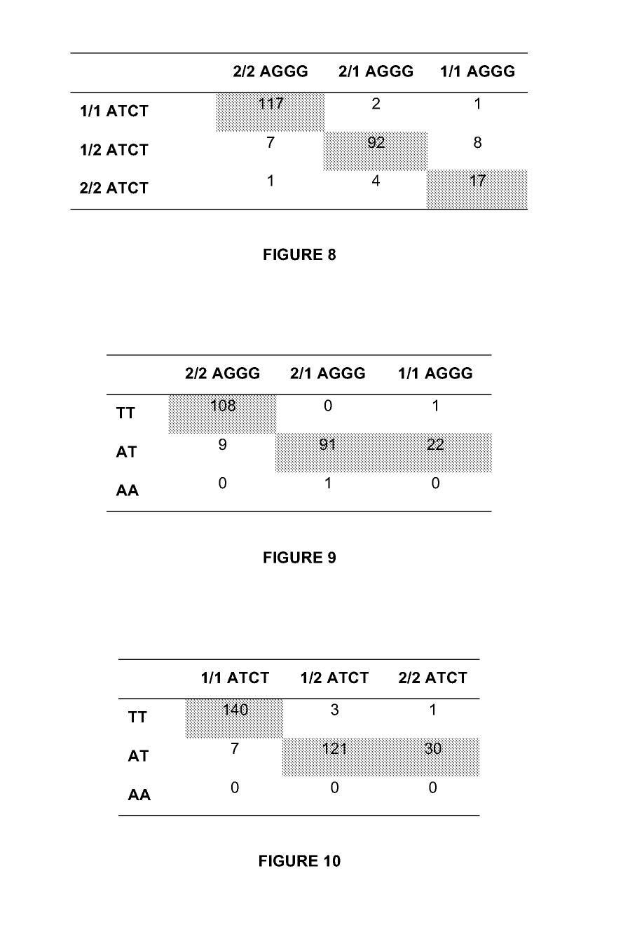

FIG. 8 shows a table of the paired genotype distributions of the COL5A1 3′-UTR -/AGGG (rs71746744) and ATCT/- (rs16399) polymorphism in the pooled South African and Australian control and chronic Achilles tendinopathy.

FIG. 9 shows a table of the paired genotype distributions of the COL5A1 3′-UTR -/AGGG (rs71746744) and A/T (rs1134170) polymorphism in the pooled South African and Australian control and chronic Achilles tendinopathy.

FIG. 10 shows a table of the paired genotype distributions of the COL5A1 3′-UTR ATCT/-(rs16399) and A/T (rs1134170) polymorphism in the pooled South African and Australian control and chronic Achilles tendinopathy.

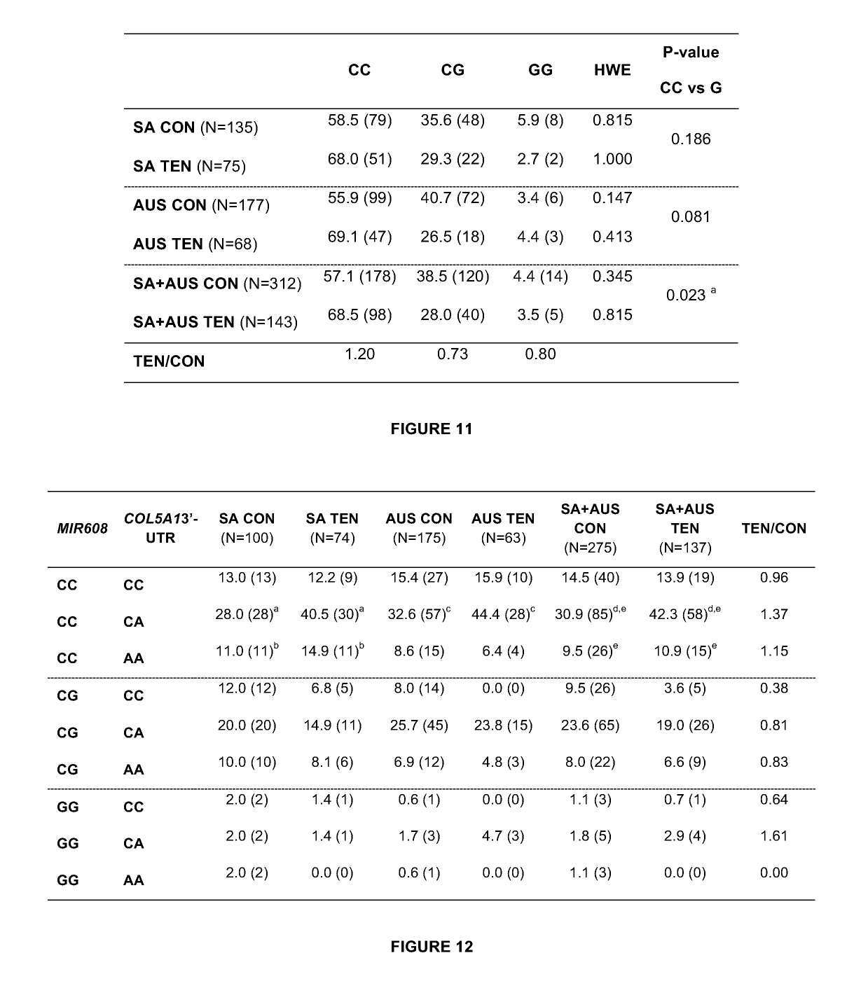

FIG. 11 shows a table summarizing genotype frequency distributions the MIR608 rs4919510 (C/G) single nucleotide polymorphism in control (CON) and chronic Achilles tendinopathy (TEN) groups of South African (SA) and Australian (AUS) cohorts, as well as the combined SA and AUS (SA+AUS) cohorts. Genotypes are expressed as percentages with numbers (N) in parenthesis. HWE are exact p-values from tests of Hardy-Weinberg equilibrium. ^{a}CC genotype vs G allele (CG+GG genotypes):odds ratio=1.6, 95% confidence interval=1.1 to 2.5

FIG. 12 shows a table summarizing the combined genotype frequency distributions of the MIR608 gene rs4919510 (C/G) single nucleotide polymorphism (SNP) and the COL5A1 3′-UTR SNP rs3196378 (C/A) within the Hsa-miR-608 binding site in control (CON) and chronic Achilles tendinopathy (TEN) groups of South African (SA) and Australian (AUS) cohorts, as well as the combined SA and AUS (SA+AUS) cohorts. Genotype pairs are expressed as percentages with numbers (N) in parenthesis. TEN/CON, SA+AUS TEN/SA+AUS CON. ^{a}SA TEN/SA CON=1.45 ^{b}SA TEN/SA CON=1.35 ^{c}AUS TEN/AUS CON=1.36 ^{d}SA+AUS TEN vs SA+AUS CON (MIR608 CC genotype+COL5A1 CC genotype), P=0.022, odds ratio=1.6, 95% confidence interval=1.1 to 2.5. ^{e}SA+AUS TEN vs SA+AUS CON (MIR608 CC genotype+COL5A1 C allele), P=0.016, odds ratio=1.7, 95% confidence interval=1.1 to 2.5.

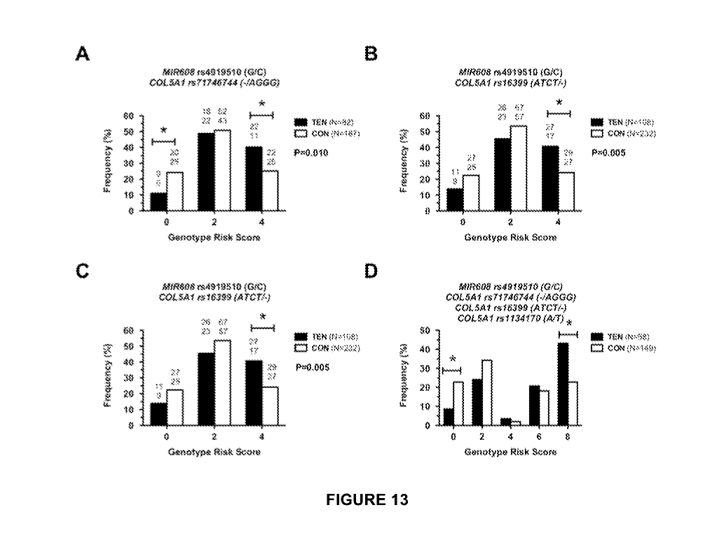

FIG. 13 shows genotype risk score frequency distributions of the Hsa-miR-608 gene (Has-miR-608) rs4919510 (C/G) single nucleotide polymorphism (SNP) and the COL5A1 3′-untranslated region (UTR), (A) rs71746744 (-/AGGG) polymorphism, (B) rs16399 (ATCT/-) polymorphism, (C) rs1134170 (A/T) SNP and, (D) all three COL5A1 3′-UTR polymorphisms in the pooled South African (SA) and Australian (AUS) control (CON, clear bars) and chronic Achilles tendinopathy (TEN, solid bars) groups. The ‘at risk’ genotypes for chronic Achilles tendinopathy at each variant contributed 2 points (rs4919510, CC; rs71746744, 2/2 AGGG; rs16399, 1/1 ATCT; rs1134170, TT) towards the genotype risk scores while the non-risk genotypes (rs4919510, CG and GG; rs71746744, 1/1 AGGG and 1/2 AGGG; rs16399, 1/2 ATCT and 2/2 ATCT; rs1134170, AT and AA) contributed 0 points. (A) As indicated by an asterisks, the genotype risk score of 0 was significantly under-represented in the TEN group, P=0.013, odds ratio (OR)=2.6 and 95% confidence interval (CI)=1.2 to 5.5. The genotype risk score of 4 was however significantly over-represented in the TEN group, P=0.014, OR=2.0 and 95% CI=1.2 to 3.5. (B) As indicated by an asterisks, the genotype risk score of 0 was significantly under-represented in the TEN group, P=0.002, OR=2.2 and 95% CI=1.3 to 3.5. (C) As indicated by an asterisks, the genotype risk score of 0 was significantly under-represented in the TEN group, P=0.007, OR=2.5 and 95% CI=1.3 to 5.1. (D) As indicated by an asterisks, the genotype risk score of 0 was significantly under-represented in the TEN group, P=0.019, OR=3.1 and 95% CI=1.2 to 8.5. The genotype risk score of 8 was however significantly over-represented in the TEN group, P=0.004, OR=2.6 and 95% CI=1.3 to 4.9. The number of observations (N) from the SA (top) and AUS (bottom) are indicated above each bar in panels A to C.

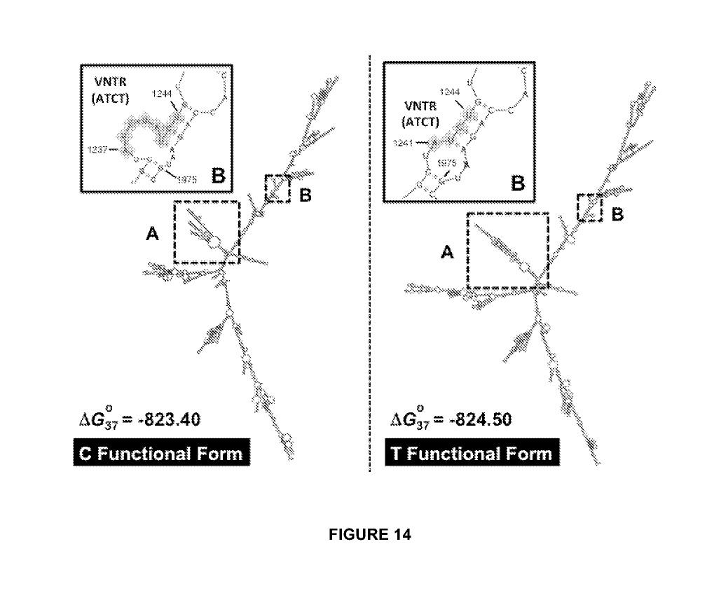

FIG. 14 shows the most stable predicted secondary structures of the C (left panel) and T (right panel) functional forms of the COL5A1 3′-UTR. The region, which contains both miRNA binding sites and the AGGG variable nucleotide tandem repeat (VNTR) (rs71746744), is indicated with box A. Box B indicated the region, which contains the ATCT VNTR (rs16399) and rs1134170 (A/T). Region B of the C (left insert) and T (right insert) functional forms of COL5A1 3′-UTR is expanded in the inserts. The two and one copies of the ATCT VNTR are highlighted in the inserted. Nucleotide positions within the 3′-UTR are also indicated. The secondary structures were generated using the S fold online RNA folding tool (available at http://sfold.wadsworth.org). The algorithm generates RNA secondary structures using a statistical sample from the Boltzmann ensemble of secondary structures. All structures were folded at 37° C. and 1M NaCl in the absence of divalent ions.

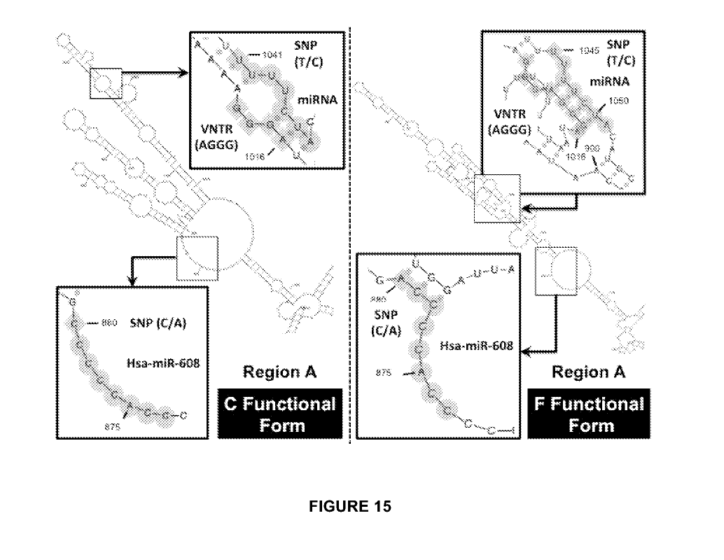

FIG. 15 shows the most stable predicted secondary structures of region A of the C (left panel) and T (right panel) functional forms of the COL5A1 3′-UTR. This region contains both polymorphic miRNA binding sites, the AGGG variable nucleotide tandem repeat (VNTR) (rs71746744), single nucleotide polymorphism (SNP) rs11103544 (T/C) and SNP rs3196378 (C/A). The region to which Hsa-miR-608 (bottom inserts) and the second unknown miRNA (top inserts) binds are expanded in the boxed inserts. The one and two copies of the AGGG VNTR are highlighted with grey diamonds in the top inserts. The miRNA binding sites are highlighted with grey circles. The SNPs within these binding sites are indicated with grey diamonds. Nucleotide positions within the 3′-UTR are also indicated. The secondary structures were generated using the Sfold online RNA folding tool (available at http://sfold.wadsworth.org). The algorithm generates RNA secondary structures using a statistical sample from the Boltzmann ensemble of secondary structures. All structures were folded at 37° C. and 1M NaCl in the absence of divalent ions.

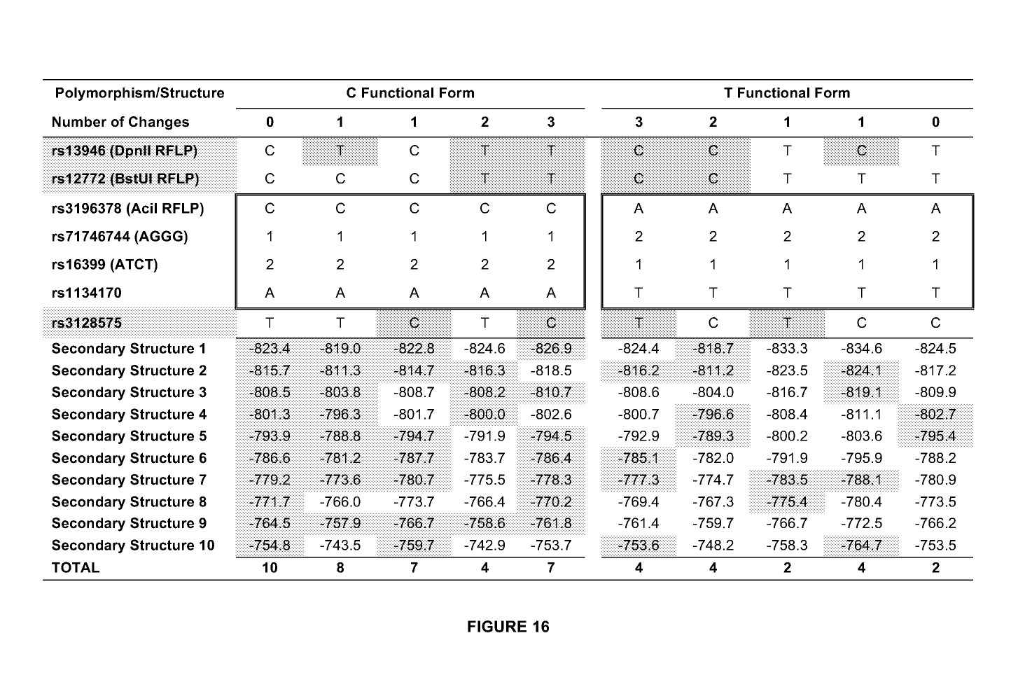

FIG. 16 shows a table summarizing the predicted secondary structures of the in silico site-directed mutated C and T functional forms of the COL5A1 3′-UTR. The seven polymorphic sites that determine the distinct C and T functional forms are indicated. The sequence associated with a specific functional form is highlighted in white, while the mutated polymorphism is highlighted in grey. The number of changes are also indicated. The algorithm generates RNA secondary structures using a statistical sample from the Boltzmann ensemble of secondary structures. All structures were folded at 37° C. and 1M NaCl in the absence of divalent ions. The ΔG values for the 10 most stable structures are indicated. The secondary structures that are similar to the C functional form of the COL5A1 3′-UTR are highlighted in grey. Major deviations from the C-functional form structure are highlighted in white. The number of secondary structures similar to the C-form for mutant generated is also indicated.

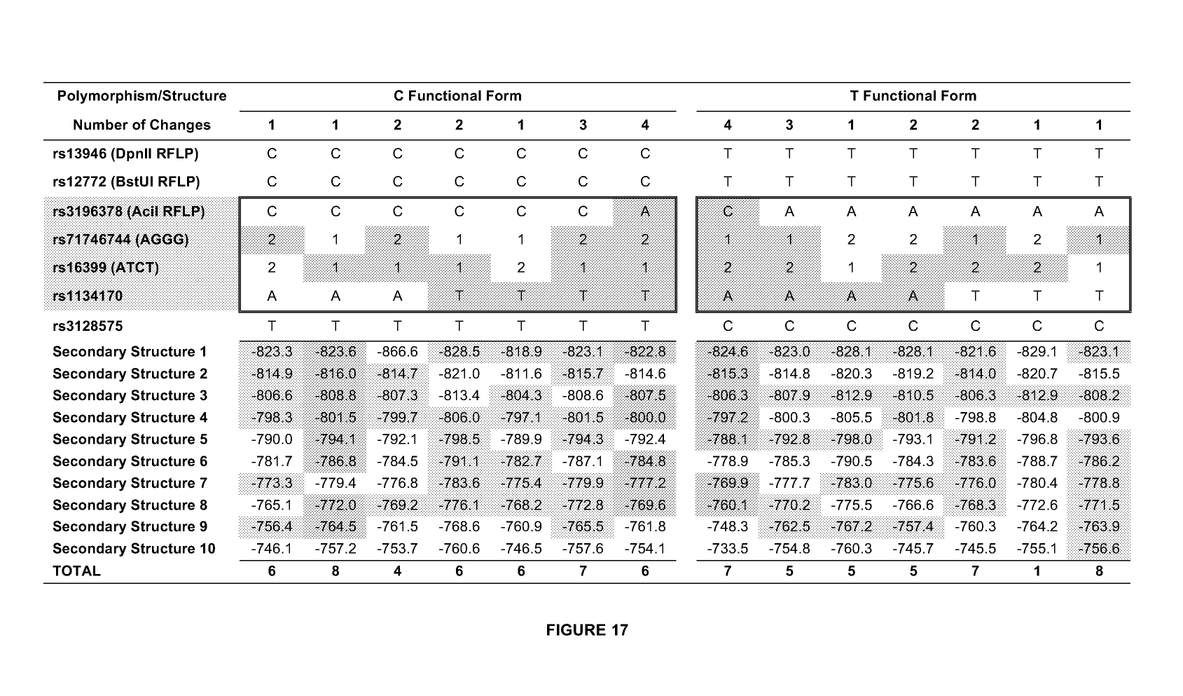

FIG. 17 shows a table summarizing the predicted secondary structures of the in silico site-directed mutated C and T functional forms of the COL5A1 3′-UTR. The seven polymorphic sites that determine the distinct C and T functional forms are indicated. The sequence associated with a specific functional form is highlighted in white, while the mutated polymorphism is highlighted in grey. The number of changes are also indicated. The algorithm generates RNA secondary structures using a statistical sample from the Boltzmann ensemble of secondary structures. All structures were folded at 37° C. and 1M NaCl in the absence of divalent ions. The ΔG values for the 10 most stable structures are indicated. The secondary structures that are similar to the C functional form of the COL5A1 3′-UTR are highlighted in grey. Major deviations from the C-functional form structure are highlighted in white. The number of secondary structures similar to the C-form for mutant generated is also indicated.

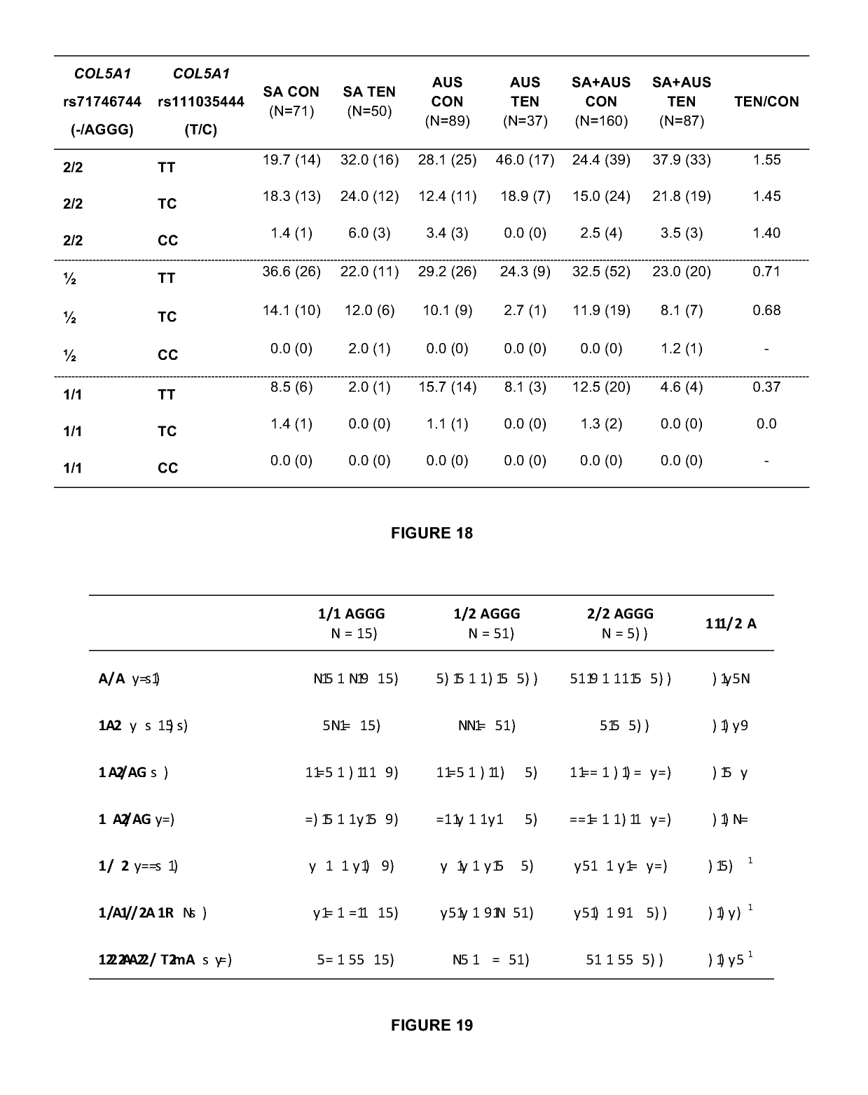

FIG. 18 shows a table summarizing the combined genotype frequency distributions of the rs71746744 (-/AGGG) and the rs71746744 (T/C, MboII RFLP) polymorphisms within the COL5A1 3′-untranslated region in control (CON) and chronic Achilles tendinopathy (TEN) groups of South African (SA) and Australian (AUS) cohorts, as well as the combined SA and AUS (SA+AUS) cohorts. Genotype pairs are expressed as percentages with numbers (N) in parenthesis.

FIG. 19 shows a table of the general characteristics, mean pre-race SR ROM and race performance of the Caucasian Two Oceans 56 km ultra-marathon athletes grouped by the three COL5A1 rs71746744 genotypes (1 AGGG/1 AGGG, 1 AGGG/2 AGGG and 2 AGGG/2 AGGG). BMI—body mass index; SR ROM—sit and reach range of motion; m—meters; min—minutes; kg—kilograms; cm—centimeters ^{a}co-varied for sex. Age, height, weight, BMI, SR ROM and finishing time are represented as a mean±standard deviation, whereas sex is represented as a percentage of males. The number of participants (N) is enclosed in parentheses.

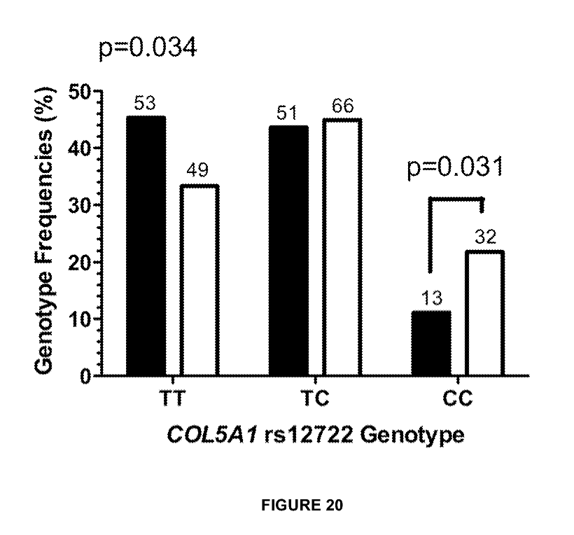

FIG. 20 shows a graph of the COL5A1 rs12722 genotype frequencies for the participants that reported a history of exercise-associated muscle cramps (EAMC) within 12 months prior to an ultra-endurance event (black bars) and those with no self-reported history of previous (lifelong) EAMC (white bars). Numbers of participants (n) are indicated above each specific column. The overall p-value is indicated above the figure, while the p-value above the genotype group refer to the pairwise post-hoc analysis.

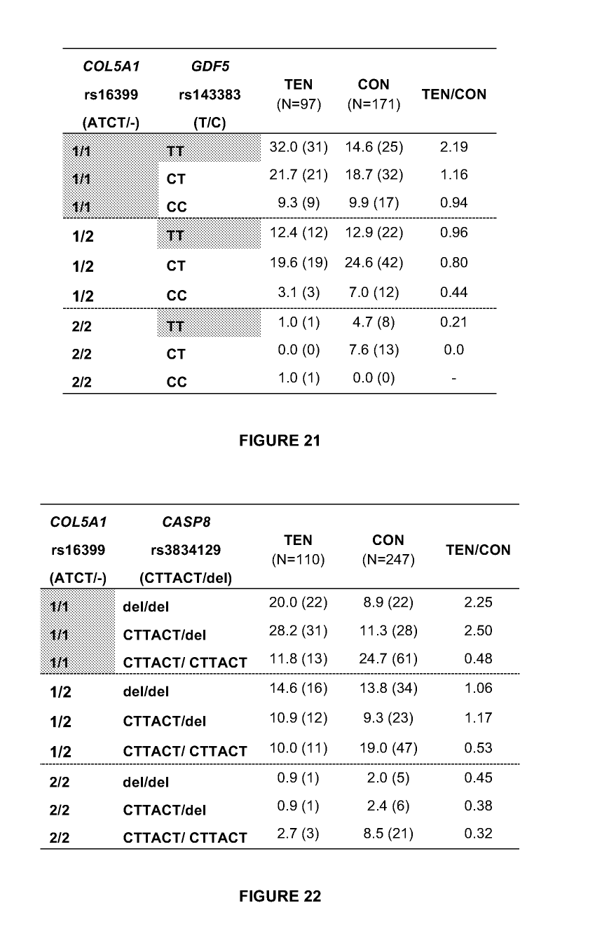

FIG. 21 shows a table of the combined genotype frequency distributions of the rs16399 (ATCT/-) VNTR within the COL5A1 3′-untranslated region and the rs143383 (T/C) polymorphism within GDF5 in combined South African and Australian control (CON) and chronic Achilles tendinopathy (TEN) cohorts. Genotype pairs are expressed as percentages with numbers (N) in parenthesis. 1/1 ATCT and TT genotypes vs rest of the genotypes: P=0.001, OR=2.7, 95% CI=1.5 to 5.0.

FIG. 22 shows a table of the combined genotype frequency distributions of the rs16399 (ATCT/-) VNTR within the COL5A1 3′-untranslated region and the rs3834129 (CTTACT/del) polymorphism within CASP8 in combined South African and Australian control (CON) and chronic Achilles tendinopathy (TEN) cohorts. Genotype pairs are expressed as percentages with numbers (N) in parenthesis. 1/1 ATCT genotype and del allele vs rest of the genotypes: P<0.001, OR=3.7, 95% CI=2.6 to 6.0.

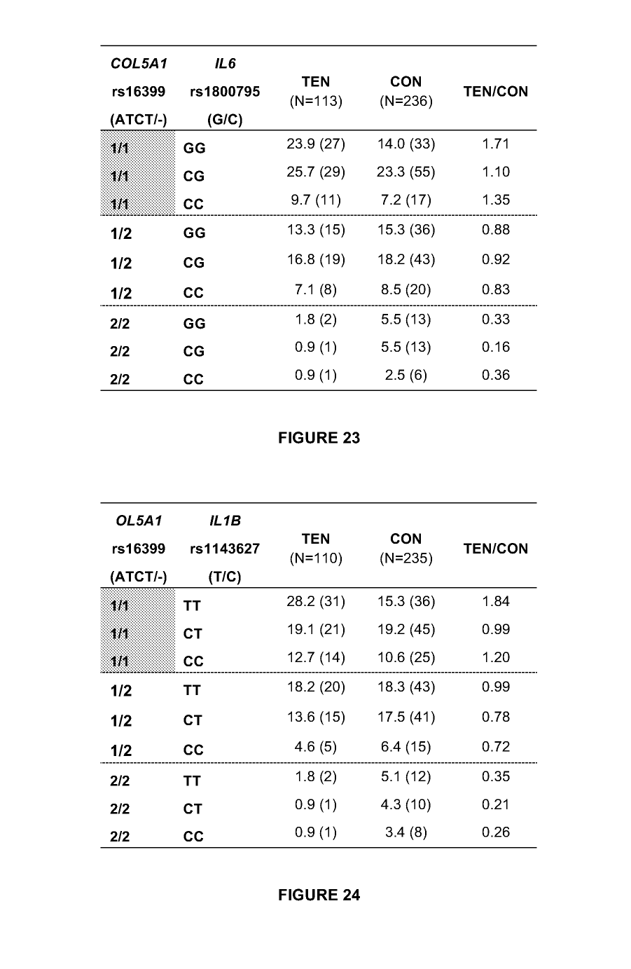

FIG. 23 shows a table of combined genotype frequency distributions of the rs16399 (ATCT/-) VNTR within the COL5A1 3′-untranslated region and the rs1800795 (G/C) polymorphism within IL6 in combined South African and Australian control (CON) and chronic Achilles tendinopathy (TEN) cohorts. Genotype pairs are expressed as percentages with numbers (N) in parenthesis.

FIG. 24 shows a table of the combined genotype frequency distributions of the rs16399 (ATCT/-) VNTR within the COL5A1 3′-untranslated region and the rs1143627 (T/C) polymorphism within IL1B in combined South African and Australian control (CON) and chronic Achilles tendinopathy (TEN) cohorts. 1/1 ATCT and TT genotypes vs rest of the genotypes: P=0.008, OR=2.2, 95% CI=1.3 to 3.7.

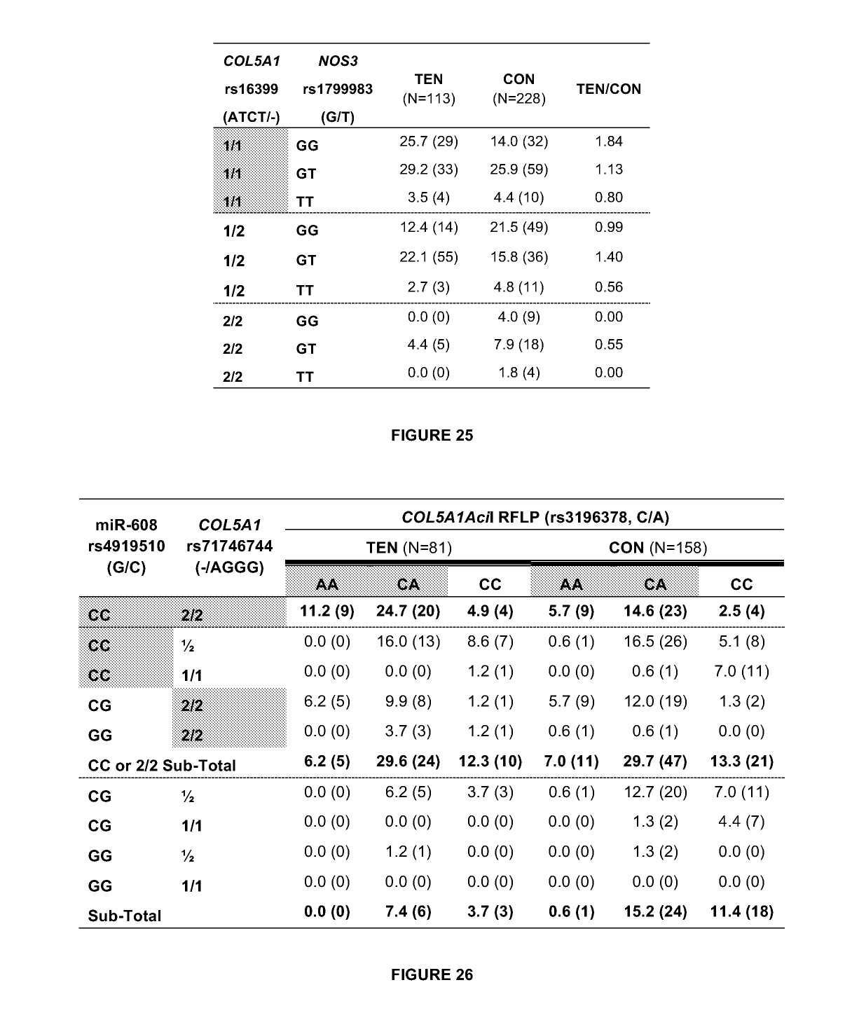

FIG. 25 shows a table of combined genotype frequency distributions of the rs16399 (ATCT/-) VNTR within the COL5A1 3′-untranslated region and the rs1799983 (G/T) polymorphism within NOS3 in combined South African and Australian control (CON) and chronic Achilles tendinopathy (TEN) cohorts. Genotype pairs are expressed as percentages with numbers (N) in parenthesis.

FIG. 26 shows that combined genotype frequency distributions of the Hsa-miR-608 gene (miR-608) rs4919510 (C/G) single nucleotide polymorphism, the COL5A1 3′-untranslated region (UTR)rs71746744 (-/AGGG) polymorphism and the AciI RFLP (C/A, rs3196378) within the Hsa-miR-608 binding site of the COL5A1 3′-UTR in the South African (SA) and Australian (AUS) combined control (CON) and chronic Achilles tendinopathy (TEN) groups. Genotype combinations are expressed as percentages with numbers (N) in parenthesis.

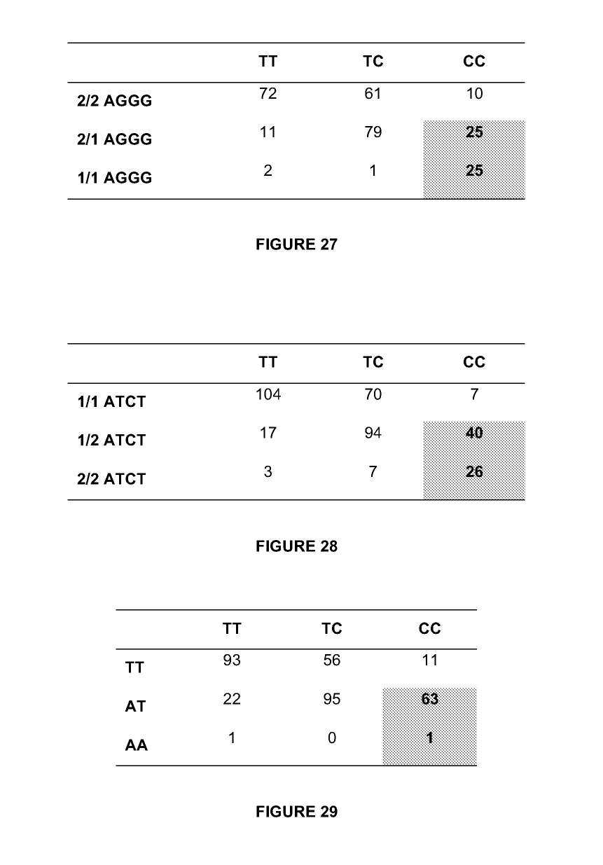

FIG. 27 shows a table of the paired genotype distributions of the COL5A1 3′-UTR T/C (rs12722, BstUI RFLP) and -/AGGG (rs71746744).

FIG. 28 shows a table of the paired genotype distributions of the COL5A1 3′-UTR T/C (rs12722, BstUI RFLP) and ATCT/- (rs16399).

FIG. 29 shows a table of the paired genotype distributions of the COL5A1 3′-UTR T/C (rs12722, BstUI RFLP) and A/T (rs1134170).

SEQ. ID. NO. 1: is the forward primer for COL5A1 (A/T) rs1134170;

SEQ. ID. NO. 2: is the reverse primer for COL5A1 (A/T) rs1134170;

SEQ. ID. NO. 3: is the forward primer for COL5A1 (-/AGGG) rs71746744;

SEQ. ID. NO. 4: is the reverse primer for COL5A1 (-/AGGG) rs71746744;

SEQ. ID. NO. 5: is the forward primer for IL-1β (T>C) (rs1143627);

SEQ. ID. NO. 6: is the reverse primer for IL-1β (T>C) (rs1143627);

SEQ. ID. NO. 7: is the forward primer for IL-6 (G/C) (rs1800795);

SEQ. ID. NO. 8: is the reverse primer for IL-6 (G/C) (rs1800795);

SEQ. ID. NO. 9: is the forward primer for COL5A1 (ATCT/-) rs16399;

SEQ. ID. NO. 10: is the reverse primer for COL5A1 (ATCT/-) rs16399;

SEQ. ID. NO. 11: is the forward primer for CASP8 (CTTACT/del) (rs3834129);

SEQ. ID. NO. 12: is the reverse primer for CASP8 (CTTACT/del) (rs3834129);

SEQ. ID. NO. 13: is the forward primer for CASP8 (G/C) D302H (rs1045485);

SEQ. ID. NO. 14: is the reverse primer for CASP8 (G/C) D302H (rs1045485);

SEQ. ID. NO. 15: is the forward primer for IL-1β (C/T) (rs16944);

SEQ. ID. NO. 16: is the reverse primer for IL-1β (C/T) (rs16944);

SEQ. ID. NO. 17: is the forward primer for GDF5 (T/C) (rs143383);

SEQ. ID. NO. 18: is the reverse primer for GDF5 (T/C) (rs143383);

SEQ. ID. NO. 19: is the sequence of Has-miR-608 with a C at the 22^{nd} position;

SEQ. ID. NO. 20: is the sequence of Has-miR-608 with a G at the 22^{nd} position.

SEQ. ID. NO. 21: is the sequence of the MiR608 gene (ENSE00001499827); and

SEQ. ID. NO. 22: is the sequence of the rs4919510 polymorphism.

### DETAILED DESCRIPTION OF AN EMBODIMENT OF THE INVENTION

For the purposes of this specification, a “polymorphism” may include a change or difference between two related nucleic acids. A “nucleotide polymorphism” refers to a nucleotide which is different in one sequence when compared to a related sequence when the two nucleic acids are aligned for maximal correspondence. A “probe” or “molecular marker” is an RNA sequence(s) or DNA sequence(s) or analogues, modified versions, or the complement of the sequences shown. This may include a “genetic marker”, which is a region on a genomic nucleic acid mapped by a molecular marker or probe. A “probe” is a composition labeled with a detectable label. A “probe” is typically used herein to identify a marker nucleic acid. A polynucleotide probe is usually a single-stranded nucleic acid sequence that can be used to identify complementary nucleic acid sequences, or may be a double- or higher order-stranded nucleic acid sequence which can be used to bind to, or associate with, a target sequence or area, generally following denaturing. The sequence of the polynucleotide probe may or may not be known. An RNA probe may hybridize with its corresponding DNA gene, or to a complementary RNA, or to other type of nucleic acid molecules. As used herein the term “functional discriminatory truncations” mean nucleic acid sequences, modified nucleic acid sequences, or other nucleic acid variants which, although they are truncated forms of sequences presented herein or variants thereof, can still bind in a discriminatory manner to target gene or nucleic acid sequences described herein and forming part of the present invention. The terms “isolated” or “biologically pure” refer to material which is substantially or essentially free from components which normally accompany it as found in its native state. An “amplified mixture” of nucleic acids includes multiple copies of more than one (and generally several) nucleic acids. “Stringent hybridization conditions” in the context of nucleic acid hybridization are sequence dependent and are different under different environmental parameters. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Highly stringent conditions are selected to be equal to the Tₘ point for a particular probe. An example of stringent wash conditions for, say, a Southern blot of such nucleic acids is a 0.2×SSC wash at 65° C. for 15 minutes. Such a high stringency wash may be preceded by a low stringency wash to remove background probe signal. An example of a low stringency wash is 2×SSC at 40° C. for 15 minutes. In general, a signal to noise ratio of 2× (or higher) than that observed for an unrelated probe in the particular hybridization assay indicates detection of a specific hybridization event. For highly specific hybridization strategies such as allele-specific hybridization, an allele-specific probe is usually hybridized to a marker nucleic acid (e.g., a genomic nucleic acid, an amplicon, or the like) comprising a polymorphic nucleotide under highly stringent conditions.

### Example 1

### Apoptosis Studies

### Methods of Apoptosis Studies

Briefly, a total of 358 unaffected control (CON) participants [159 South Africa (SA CON) and 199 Australia (AUS CON)] and 166 affected AT (TEN) participants (87 SA TEN and 79 AUS TEN) were genotyped for the variants CASP8 (rs384129) and CASP8 (rs1045485). Logistic regression was used to derive risk models for AT. A receiver operator characteristic (ROC) curve was plotted to determine the effectiveness of a model to capture TEN risk. This study indicates the independent association of CASP8_rs1045485 and CASP8_rs3834129 as well as their haplotype with TEN risk and the identification of an optimal model which included genetic loci CASP8_rs384129 and CASP8_rs1045485 together with gender to capture TEN risk in both SA and AUS.

### Participants

The South African (SA) and Australian (AUS) participants were of self-reported European Caucasian ancestry. A total of 159 asymptomatic control participants (designated as SA CON) and 87 with diagnosed Achilles tendinopathy (designated as SA TEN) together with 199 asymptomatic control participants (designated as AUS CON) and 79 diagnosed Achilles tendinopathy (designated as AUS TEN) participants were recruited for the study as previously described (Mokone et al Am J Sports Med 2005; 33:1016-1021; Mokone et al. Scand J Med Sci Sports 2006; 16:19-26; September et al. Br J Sports Med 2011; 45:1040-1047; September et al. Int J Sports Med 2008; 29:257-263; September et al. Br J Sports Med 2009; 43:357-365).

Participants signed informed consent forms according to the Declaration of Helsinki, provided personal particulars and completed a questionnaire regarding medical history (September et al. Int J Sports Med 2008; 29:257-263). Approval for the study was obtained from the Research Ethics Committee of the Faculty of Health Sciences, The University of Cape Town (reference number 172/2005) and Human Ethics Committee of La Trobe and Deakin Universities, Melbourne, Australia.

### DNA Extraction

DNA was extracted for all participants as previously described (Mokone et al Am J Sports Med 2005; 33:1016-1021; September et al. Br J Sports Med 2009; 43:357-365).

### Genotyping

CASP8 (Srivastava et al. Mol Carcinog 2010; 49:684-692) rs3834129 and rs1045485 were investigated. Genotyping of rs384129, rs1045485 and rs2779249 was conducted using the Taqman method according to standard techniques and rs1799983 was genotyped using restriction fragment length polymorphism analysis.

### Statistics

Basic characteristics of the study groups were presented and summarized previously (Mokone et al Am J Sports Med 2005; 33:1016-1021; Mokone et al. Scand J Med Sci Sports 2006; 16:19-26; September et al. Br J Sports Med 2011; 45:1040-1047; September et al. Int J Sports Med 2008; 29:257-263; September et al. Br J Sports Med 2009; 43:357-365; Fu et al. J Hypertens 2009; 27:991-1000).

The relationship between the genotypes and AT susceptibility was tested and found not to differ significantly between the countries. The data from the population groups were combined for all further analyses. Age, gender, country and whether the individual was born in the specific country were considered confounders and were adjusted for in all analyses by including them in the models as fixed effects. Logistic regression was used to compare the TEN and CON groups, as well as the countries with respect to genotype, allele and allele-combination frequencies. Significant genotype associations were further examined to determine whether it was the result of heterozygote, recessive or a dominant effect, by recoding the genotypes appropriately in the logistic regression models. Haplotype and allele combination associations were tested for additive, dominant and recessive models on the logit scale.

### Inflammatory Risk Model for AT

Logistic regression was used to derive risk models for AT. Three models were constructed; the first incorporated the four known confounders and the genotypes at the four loci implicated in the apoptosis signalling cascade (rs384129, rs1045485, rs1799983, 2779249), The second contained the same factors as the first, plus the interleukin loci previously genotyped (rs1800795; rs16944; rs1143627). The optimal model was backwards selected from the first, using Akaike criterion.

A receiver operating characteristic (ROC) curve¹⁸ was constructed for each of the three logistic regression models to compare the effectiveness of each model to predict TEN risk. The area under the ROC curve (AUC) was used to quantify the overall ability of the model to discriminate between diagnostic groups based on genotype risk.

Results corresponding to a p-value of less than 0.05 were described as significant. The programming environment, R (R Development Core Team. R: A language and environment for statistical computing. R Foundation for Statistical Computing, Vienna, Austria 2010.) and R packages were used for all analyses. The R package, genetics (Warnes et al. R package version 1 3 4 2008) was used to estimate genotype and allele frequencies and Hardy-Weinberg equilibrium probabilities. Frequencies of allele combinations were inferred and analysed using the R package, haplo.stats. (Schaid et al. Am J Hum Genet 2002; 70:425-434; Sinnwell et al. R package version 1 4 4 2009). ROC curves were created using the R package Epi (Carstensen et al. R package version 1.1.20.2011)

### Results of Apoptosis Studies

### Genotype and Allele Frequency Distributions

Genotype and minor allele frequency distributions for each of the polymorphisms together with the HWE p-values are shown in FIG. 1.

CASP8 rs3834129

A significant difference in the genotype (p=0.0294) but not the allelic distribution (p=0.2528), was detected for rs3834129 between the CON and TEN groups, after adjusting for the confounders. A heterozygote advantage model provided the best fit (OR=0.61; p=0.0141; 95% CI: 0.40-0.90); the odds of TEN with D/I is 39% less than the odds with either (I/I or D/D) homozygote. A dominant model for the minor allele (I) also provided a significant fit (OR=0.60; p=0.0215; 95% CI: 0.38-0.93); with a D/D genotype the odds of TEN is 67% more (OR=1.67; CI 1.08 to 2.60) than the D/I or I/I genotypes. The distribution for rs3834129 were similar between SA and AUS (p=0.860 and p=0.3578) after adjusting for the confounders.

CASP8 rs1045485

Highly significant differences in the genotype (p=0.0009) and allele (p=0.00027) distributions were detected for rs1045485 between the two countries after adjusting for the confounders. Significant differences were detected in the genotype (p=0.0213) and allele (p=0.0097) distributions between the CON and TEN groups, after adjusting for the confounders. The highly significant allelic effect can be interpreted as: Each C allele reduces the odds of TEN by 41% (OR=0.59; 95% CI: 0.39-0.87). Investigating the significant genotype effect showed two transmission models with highly significant fits, the heterozygote advantage (only the G/C genotype) reduces the odds of TEN (OR=0.56; p=0.0094; 95% CI: 0.35-0.86) compared to both homozygotes; and the dominant model (any minor C allele) either C/C or C/G genotype reduces the odds of TEN (OR=0.55; p=0.0065; 95% CI: 0.35-0.84) compared to G/G.

### Interactions: Allele Combinations

Frequencies were inferred for the allele combinations rs384129, rs1045485, rs1799983 and rs2779249. The most common allele combinations were D-G-G-C (CON=17%; TEN=21%) and I-G-G-C (CON=18%; TEN 16%); whereas both I-C-T-A and I-C-T-C were detected at frequencies below 1% in CON. The four-way allelic combination was not significantly associated with AT susceptibility after adjusting for the confounders and similarly nor were any of the 3-way allelic combinations.

The CASP8 inferred haplotype was significantly associated with AT risk for additive (p=0.0210), dominant (p=0.0052) and recessive (p=0.0036) allelic combination models. The D-C inferred allele combination was present in 15% of CON and 9% of TEN and showed a dominant protective effect such that an individual needs only one of those combinations to be protected against AT. While the D-G inferred allele combination was present in 35% of CON and 45% of TEN; showing a recessive risk effect such that you need to be homozygous for D-G allele combination to be at increased risk of AT.

### Inflammatory Risk Models for Achilles Tendinopathy

FIG. 2 A shows a ROC curve of the model containing the 4 known confounders and the genotype data from rs384129, rs1045485; rs1799983 and rs2779249 to predict AT risk; AUC=0.684 (132 TEN; 281 CON); and sensitivity=62.9%, and specificity=66.2%.

The model which contained genotypes for rs18000795, rs16944, rs1143627, rs384129, rs045485, rs1799983 and rs2779249) and the confounders had an AUC=0.705 (244 CON and 116 TEN); sensitivity=45.7 and specificity=84%.

The factors which jointly contributed to the optimal model for evaluating risk assessment of AT were the genetic loci rs384129 and rs1045485, and gender (FIG. 3); the AUC=0.667 (151 TEN and 336 CON) (FIG. 2.B); sensitivity=60.9% and specificity=64.3%.

### Discussion of Apoptosis Studies

The inventors have surprising found an association of the CASP8 polymorphisms and their haplotype, and have identified an apoptosis polygenic profile for indicating an increased risk of AT.

The recessive model for rs3834129 suggests that individuals with a D/D genotype have a 68% higher risk of AT than those with either I/I or D/I genotypes. This finding is unexpected since the del allele destroys a Sp1 binding element which results in decreased caspase-8 expression (Sun et al. Nat Genet 2007; 39:605-613). Reduced caspase-8 expression was expected to protect against excessive apoptosis and the deletion allele was therefore expected to protect against AT. Interestingly, the heterozygote advantage model predicts that subjects that are heterozygous, D/I genotype, at this locus have a reduced risk compared to subjects having either homozygote (D/D and I/I).

At the rs1045485 locus, there was a highly significant protective effect of the heterozygote genotype, C/G compared to G/G genotypes. The C/C genotype was rare (2%) so it was not surprising that a heterozygote (G/C reduces AT risk by 44% compared to G/G+C/C) and an additive allelic (each C allele reduces risk by 41%) and a dominant model (C/C+C/G reduces AT risk by 45% compared to G/G) all provided highly significant fits.

The CASP8 polymorphism associations were mirrored in the CASP8 haplotype. The CASP8 D-C haplotype was associated with reduced AT risk in the additive, dominant and recessive allelic combination models. Genotyping other SNPs in the region implicated by the haplotype may provide more informative haplotypes in identifying the critical casual region.

Lastly, this study suggests that the more biomarkers incorporated into the design of a risk profile, the greater the effectiveness to predict risk, as would be expected in a polygenic condition (AUC=0.705). A preferred risk model suggests that the two loci CASP8 together with gender is sufficient to predict AT risk (AUC=0.667).

Another preferred model estimates that the minimum risk for AT occurs in females who are homozygous C/C and heterozygous D/I for rs384129 and rs045485 respectively and on the contrary males with the G/G and D/D genotypes at the two CASP8 loci are at maximum risk for AT. Although all inferred allele combinations were not significantly associated with AT risk, the ROC analysis suggests that the loci are collectively able to discriminate between affected and unaffected individuals. This suggests that the cumulative effect of these protein products contribute to AT risk.

Collectively, these results further implicate the apoptosis signalling cascade as one of the biological pathways involved in the development of AT. The associations observed in this study should be explored in larger independent groups to elucidate the biological significance of the apoptosis signalling cascade in musculoskeletal soft tissue injuries.

### Example 2

### COL5A13′-UTR and MIR608 Studies

### Methods of COL5A13′-UTR and MIR608 Studies

### Participants

Three hundred and forty-two asymptomatic control participants (CON) and 160 with diagnosed Achilles tendinopathy (TEN) were included in this study. The TEN and CON participants were recruited from South African (SA TEN, N=81 and SA CON=149) and Australia (AUS TEN, N=79 and AUS CON=193) as previously described Mokone et al. Am J Sports Med 2005; 33:1016-1021, Mokone et al. Scand J Med Sci Sports 2006; 16:19-26, September et al. Br J Sports Med 2009; 43:357-365, September et al. Br J Sports Med 2011; 45:1040-1047). All participants were of self-reported European Caucasian ancestry. The profiles of the CON and TEN SA and AUS participants were previously described in detail (September et al. Br J Sports Med 2011; 45:1040-1047).

All the participants signed an informed consent form prior to participation in this study. Approval for the study was obtained from the Human Research Ethics Committee of the Faculty of Health Sciences at the University of Cape Town and the Human Ethics Committee of La Trobe and Deakin Universities, Melbourne, Australia.

### DNA Extraction

DNA was extracted for the SA (Mokone et al. Am J Sports Med 2005) and AUS participants as previously described (September et al. Br J Sports Med 2009; 43:357-365).

### COL5A1 3′-UTR Genotyping

The participants were genotyped for the rs4919510 (-/AGGG; 91 TEN and 198 CON), rs16399 (ATCT/-; 120 TEN and 254 CON) and rs1134170 (A/T; 107 TEN and 241 CON) polymorphisms within the 3′-UTR of the COL5A1 gene. Genotyping was performed using custom designed Fluorescence-based Taqman® PCR assays (Applied Biosystems, Foster City, Calif., USA). Allele specific probes and flanking primer sets (sequences available on request) were used along with a pre-made PCR mastermix containing ampliTaq® DNA polymerase Gold (Applied Biosystems, Foster City, Calif., USA) in a final reaction volume of 8 μl. The two-step PCR consisted of a 10 min heat activation step (95° C.) followed by 40 cycles of 15s at 92° C. and 1 min at 60° C. using the XP Thermal Cycler, Block model XP-G (BIOER Technology CO., LTD, Tokyo, Japan). End-point fluorescence using a 7900 HT Fast Real-Time PCR System and the SDS Software version 2.3 (Applied Biosystems, Foster City, Calif., USA) was used to determine the genotypes of each polymorphism.

In addition, high resolution melting (HRM) analysis was performed for rs16399 (ATCT/-) by the Central Analytical Facility (University of Stellenbosch, Stellenbosch, South Africa). A designed primer set (FWD: 5′ CAC TTC TCT CTT GTG GCT C 3′, REV: 5′ CAG TGC GCC TTC AAG GAG AC 3′) was used for that purpose. DNA template was quantified using The NanoDrop ND1000 (NanoDrop Technologies, Wilmington, Del., USA) and normalized to 5 ng/μl. Reactions were set up in an ABI Fast 96-well optical plate (Applied Biosystems, Foster City, Calif., USA) using the following reaction: 1×ABI MeltDoctor HRM Master Mix (Applied Biosystems, Foster City, Calif., USA), 6 pmol of each primer, 20 ng of DNA template with a final volume of 20 μl. The HRM-PCR was performed in the StepOne Real-time PCR System (Applied Biosystems, Foster City, Calif., USA) with the following cycling and melting conditions: An activation step at 95° C. for 10 mins followed by 40 cycles with a denaturing step at 95° C. for 15 sec and annealing step at 60° C. for 1 min. This was followed by a melt curve comprising the sequential steps: a denaturing step at 95° C. for 10 sec, an annealing step at 60° C. for 1 min, a HRM step at 95° C. for 15 sec (ramping rate of 1%) ending with an annealing step at 60° C. for 15 sec. Sequenced controls representative of each genotype were included in each experiment. Data collection and primary analysis including amplification plots were performed with StepOne Software Version 2.2.1 (Applied Biosystems, Foster City, Calif., USA). The high-resolution melt analysis was performed using the High Resolution Melt Software Version 3.0.1 (Applied Biosystems, Foster City, Calif., USA). Variants were called automatically and the pre-melt region was between 70.9° C. and 71.3° C. while the post-melt region was between 78.0° C. and 78.3° C. Aligned melt curves and difference plots are generated as well as silhouette scores for each sample. Any samples with low amplification or with outlier melt profiles were removed from the HRM analysis.

### MIR608 Genotyping

The participants (143 TEN and 312 CON) were genotyped for the G>C SNP (rs4919510) present in the MIR608 geneusing a custom designed Fluorescence-based Taqman® polymerase chain reaction (PCR) assay (Applied Biosystems, Foster City, Calif., USA) as described above. The mature Hsa-miR-608 has the following sequence: 5′-AGGGGTGGTGTTGGGACAGCT SCG T-3′, where S is a C or G.

mRNA Secondary Structure and Binding Energy

All secondary structures of the wild-type and mutated C and T functional forms of the COL5A1 3′-UTR were generated using the Sfold online RNA folding tool (available at http://sfold.wadsworth.org) (Ding, et al. Nucleic acids research 2003, 31(24), 7280-7301; Ding et al. RNA 2005 (New York, N.Y.), 11(8), 1157-1166). The Sfold RNA folding algorithm generates RNA secondary structures using a statistical sample from the Boltzmann ensemble of secondary structures. All structures are folded at 37° C. and 1M NaCl in the absence of divalent ions.

The predicted change in Gibb's Free Energy in the Hsa-miR-608:COL5A1mRNA complex was predicted using the miRanda algorithm (v3.0) (http://cbio.mskcc.org/microrna_data/miRanda-aug2010.tar.gz-) (Enright et al. Genome Biology, 2003 5(1), R1. doi:10.1186/gb-2003-5-1-r1).

### Statistics

Data were analysed using STATISTICA Version 10.0(StatSoft, Tulsa, Okla., USA) and GraphPad Prism version 5.0d for Mac OS X (GraphPad Software, San Diego, Calif., USA, www.graphpad.com) programs. A one-way analysis of variance was used to determine any significant differences between the characteristics of the TEN and CON groups within the AUS and SA cohorts. A Chi²-analysis or Fisher's exact test was used to analyse any differences in the genotype frequencies and other categorical data between the groups. Significance was accepted when P<0.05 and P<0.025 when combined gene-gene interactions or effects were analysed. Combined genotype frequencies were analyzed using the Monte Carlo test (CLUMP program, version 2.0) (Sham et al. Ann Hum Genet. 1995; 59:97-105). Hardy-Weinberg equilibrium (HWE) was established using the program Genepop web version 3.4 (http://genepop.curtin.edu.au/). Linkage disequilibrium (LD) was calculated using CubeX: cubic exact solution (www.oege.org/software/cubex/) (Gaunt et al BMC bioinformatics 2007, 8, 428).

### Results of COL5A13′-UTR and MIR608 Studies

The SA and AUS TEN and CON groups used in study have been previously described in detail (September et al. Br J Sports Med 2009; 43:357-365). In summary, there were significantly more males within the combined SA and AUS TEN (73.0%, N=159) groups when compared to the combined CON groups (50.6%, N=340, P<0.001). The combined TEN and CON groups were matched for age (TEN age of initial injury 39.8±14.5 years, N=153vs CON age at recruitment 37.7±11.7 years, N=331; P=0.091) and height (TEN 176±9 cm, N=147vs CON 172±13 years, N=335; adjusted for sex P=0.960). The combined TEN groups were significantly heavier (TEN 79.6±14.1 kg, N=153vs CON 72.5±13.4 kg, N=339; adjusted for sex P=0.003; adjusted for sex and recruitment age P=0.039) and had an increased body mass index (BMI) (TEN 25.7±3.8 kg·m⁻², N=147vs CON 24.2±3.6 kg·m⁻², N=330; adjusted for sex P=0.002; adjusted for sex and recruitment age P=0.152) when compared to the combined CON groups. The combined TEN groups were however recruited on average 8.6±9.6 years (N=153) after their initial injury.

### COL5A1 3′-UTR Genotype Frequencies

FIG. 4 shows a schematic representation of the region (from SNP rs12722 to rs1134170) within the 3′-untranslated region (UTR) of the human COL5A1 gene on chromosome 9q34 associated with several exercise-associated phenotypes and the MIR608 gene on chromosome 10q24.

With the exception of significant differences between BMI and the three rs16399 (ATCT/-) genotype groups, there were no significant genetic interactions with any of the physiological variables (age, height, weight and sex) for any of the COL5A1 3′-UTR variants (results not shown). Participants with a 2/2 ATCT genotype (BMI 27.0±6.1 kg·m⁻²) were significant larger than those with a 2/1 ATCT (BMI 24.0±3.2 kg·m⁻², P value adjusted for age at recruitment and sex=0.001) or a 1/1 ATCT (BMI 24.6±3.4 kg·m⁻², P value adjusted for age at recruitment and sex=0.009) genotype.

The genotype distributions of rs71746744, rs16399 and rs1134170 were similar within the SA and AUS cohorts (FIG. 5) and were therefore combined for further analysis. The 2/2 AGGG, 1/1 ATCT and TT genotype frequencies of rs71746744, rs16399 and rs1134170 respectively were significantly over-represented in the combined SA and AUS cohorts. Except for rs1134170, the other polymorphisms were in Hardy-Weinberg equilibrium. The three polymorphisms were in linkage disequilibrium (FIGS. 6 and 7). The paired genotype distributions of the COL5A1 3′-UTR -/AGGG (rs71746744) and ATCT/- (rs16399) polymorphism is shown in FIG. 8. The paired genotype distributions of the COL5A1 3′-UTR -/AGGG (rs71746744) and A/T (rs1134170) polymorphism is shown in FIG. 9. The paired genotype distributions of the COL5A1 3′-UTR ATCT/- (rs16399) and A/T (rs1134170) polymorphism is shown in FIG. 10.

MIR608 Genotype Frequencies and Interactions with its COL5A1 3′-UTR Binding Site

There were no significant genetic interactions with any of the physiological variables for the rs4919510 polymorphism within the MIR608 gene (results not shown). The genotype distributions of rs4919510 were similar within the SA and AUS cohorts (FIG. 11) and were therefore also combined for further analysis. The CC genotype frequency was significantly over-represented when compared to the G allele (CG and GG genotypes) in the combined SA and AUS TEN group (P=0.023, OR=1.6, 95% CI=1.1 to 2.5) (FIG. 11). Polymorphism rs4919510 was in HWE in all the groups.

The Hsa-miR-608 binding site within the COL5A1 3′-UTR is polymorphic (September et al. Br J Sports Med 2009; 43:357-365). We investigated the combined genotype frequencies M/R608SNP rs4919510 and SNP rs3196378 (C/A, AciI RFLP) within the miRNA binding site. In addition, the A allele of rs3196378 was identified within the T functional form of the COL5A1 3′-UTR which was predominately cloned from TEN subjects (Laguette et al. Matrix biology: journal of the International Society for Matrix Biology, 30(5-6), 338-345. doi:10.1016/j.matbio.2011.05.001). The combined genotype distributions of rs4919510 and rs3196378 were similar within the SA and AUS cohorts (FIG. 12) and were therefore combined for further analysis. Although there were no significant differences between the groups when running Monte Carlo tests, the combined M/R608CC and COL5A1 rs3196378 CA genotypes were significantly over-represented in TEN (42.3%) when compared to the CON (30.9%) groups (P=0.022, OR=1.6, 95% CI=1.1 to 2.5). The M/R608CC and COL5A1 rs3196378 AA genotypes distributions was however similar between the TEN (9.5%) and CON (10.9%) groups. This similarity was due the combined MIR608 CC and COL5A1 CC combined genotypes being under-represented within the AUS TEN cohort, but not the SA TEN cohort (FIG. 12). The combined MIR608 CC genotype and COL5A1 rs3196378 C allele (CA and AA genotypes) were significantly over-represented in TEN (53.2%, N=73) when compared to the CON (40.4%, N=111) groups (P=0.016, OR=1.7, 95% CI=1.1 to 2.5).

The most favourable binding energy was calculated to be between the C allele of the mature Hsa-miR-608 and the C allele of its COL5A1 binding site (−24.5 kcal/mol). The least favourable calculated binding energy was between the G allele of Hsa-miR-608 and either variants (C or A) of its binding site (−22.2 kcal/mol). The binding energy between the C allele of Hsa-miR-608 and the A allele of its COL5A1 binding site was calculated to be −23.5 kcal/mol.

### MIR608 and COL5A1 3′-UTR Genotype Interactions

The CC MIR608 rs4919510, 2/2 AGGG COL5A1 rs71746744, 1/1 ATCTCOL5A1 rs16399 and TT COL5A1 rs1134170 genotypes were all independently associated with increased risk of chronic Achilles tendinopathy (FIGS. 12 and 13) and therefore gene-gene interactions were investigated. There were significantly more participants within the combined TEN groups with at risk genotypes for both rs4919510 (CC) and rs71746744 (2/2 AGGG) when compared to the combined CON groups (genotype risk score of 4, P=0.014, odds ratio=2.0, 95% confidence interval=1.2 to 3.5) (FIG. 13). The participants with none of the MIR608 or COL5A1 risk genotypes (genotype score of 0) were significantly under-represented within the combined TEN groups (FIG. 13A, rs4919510 and rs71746744, P=0.013, odds ratio=2.6, 95% confidence interval=1.2 to 5.5; FIG. 13B, rs4919510 and rs16399, P=0.002, odds ratio=2.2, 95% confidence interval=1.3 to 3.5; FIG. 13C, rs4919510 and rs1134170, P=0.007, odds ratio=2.5, 95% confidence interval=1.3 to 5.1).

The 2/2 AGGG rs71746744, 1/1 ATCT rs16399 and TT rs1134170 COL5A1 genotypes were significantly over-represented (P=0.006; odds ratio=2.3; 95% confidence interval 1.3 to 4.3) within the combined TEN participants (60.0%, N=36 of 60) when compared to the combined CON participants (39.4%, N=61 of 155). In contrast, participants with none of the three at risk COL5A1 genotypes were significantly over-represented (P=0.002; odds ratio=2.7; 95% confidence interval 1.4 to 5.0) within the CON participants (55.5%, N=86 of 155) when compared to the TEN participants (31.7%, N=19 of 60). When the CC MIR608 rs4919510 at risk genotype was included in the analyses the participants with all four risk genotypes (genotype score of 8) were significantly over-represented within the TEN participants (P=0.004; odds ratio=2.6; 95% confidence interval 1.3 to 4.9), while those with none of the four risk genotypes (genotype score of 0) was significantly under-represented within the TEN participants (P=0.019; odds ratio=3.1; 95% confidence interval 1.2 to 8.5) (FIG. 13D).

### Predicted Secondary Structures of the Major COL5A1 3′-UTR Functional Forms

There were structural differences in the most stable C and T functional forms of the COL5A1 3′-UTR (FIG. 14). Of note, the predicted secondary structure of the region which contains both miRNA binding sites (Region A in FIG. 14 and FIG. 15) were distinctly different. The AGGG VNTR, which associated with TEN, appears to be directly involved in the secondary structure of the second miRNA binding site (Top Inserts in FIG. 15).

To date, only seven polymorphic sites have been identified within a 2.5 kb region of the COL5A1 3′-UTR to influence the predicted secondary structures of the C and T functional forms. In attempted to identify which of the seven variants were responsible for determining the “gross” structural differences between the two functional forms, the secondary structures of the COL5A1 3′-UTR were determined after in silico sit-directed mutagenesis. The structure of region A (FIG. 14 and FIG. 15) were similar in all of the ten most stable predicted secondary structures identified for the C 3′-UTR. Interestingly, much more variation within the structure of region A was noted for the T form (FIGS. 17 and 18). The characteristic structure of region A within the C form was only present within 20% of the predicted T structures (structure 4 and 5, FIG. 16). As illustrated in FIGS. 16 and 17 all seven polymorphic sites probably contribute to the structural differences of region A within the C and T functional forms. Of note, was that the characteristic structure of region A within the C form was present within 80% of the predicted T structures when only a single AGGG repeat was included in the structure (structure 4 and 5, FIG. 16).

### Discussion of COL5A13′-UTR and MIR608 Studies

The first main finding of this study was that three additional sequence variants, rs71746744 (AGGG/-), rs16399 (-/ATCT) and rs1134170 (T/A), downstream from the previously associated BstUI RFLP (rs12722) within the COL5A1 3′-UTR was associated with chronic Achilles tendinopathy (refer to FIG. 4). Specifically, the 2/2 AGGG, 1/1 ATCT and TT genotypes of rs71746744, rs16399 and rs1134170, respectively, were significantly over-represented in the tendinopathy patients. There was a two-fold increased risk of developing chronic Achilles tendinopathy with any one of these three genotypes. These three sequence are tightly linked and localize within a256 bp region of the 3′-UTR, which also contains a polymorphic (SNP rs11103544, MboII RFLP) miRNA binding site. We have previously shown that a 57 bp region containing this polymorphic miRNA binding site and the AGGG VNTR was functional (Laguette et al. (2011) Matrix biology: journal of the International Society for Matrix Biology, 30(5-6), 338-345. doi:10.1016/j.matbio.2011.05.001). In addition, the functional differences between the C- and T-functional forms of the COL5A1 3′-UTR were abolished when this small 57 bp region was deleted from the entire 2.5 kb 3′-UTR, suggesting that this region contains important regulatory elements responsible for the increase in mRNA stability within the T functional form (Laguette et al. Matrix biology: journal of the International Society for Matrix Biology, 2011; 30(5-6), 338-345. doi:10.1016/j.matbio.2011.05.001). Further work is however required to identify the specific elements and the specific miRNA that bind to this putative site.

Although the miRNA binding site within this region is polymorphic we have previously shown that SNP rs11103544 (MboII RFLP) was not associated with chronic Achilles tendinopathy (September et al. Br J Sports Med 2009; 43:357-365). In addition this SNP was not one of the major sequence variants that differentiated between the C- and T-functional forms of the COL5A1 3′-UTR (Laguette et al. Matrix biology: journal of the International Society for Matrix Biology, 2011:30(5-6), 338-345. doi:10.1016/j.matbio.2011.05.001). Furthermore, investigations have concluded that SNP rs11103544 did not interact with the AGGG VNTR to modify the association with chronic Achilles tendinopathy (FIG. 18). Although SNP rs11103544 was not associated with chronic Achilles tendinopathy, the AGGG VNTR, which is 25 bp upstream of the miRNA, directly influenced the predicted secondary structure of the putative miRNA binding site (Top Inserts of FIG. 15). It is therefore tempting to speculate that the AGGG VNTR modulates miRNA binding to this site.

The second main finding of this study was that the polymorphic MIR608 gene (SNP rs4919510) was also associated with chronic Achilles tendinopathy. The CC genotype of this variant was significantly over-represented within the Tendiopathic participants. The MIR608 gene encodes for miRNA, Hsa-miR-608, which binds to a functional polymorphic cis-acting element within the COL5A1 3′-UTR (September et al. Br J Sports Med 2009; 43:357-365; Laguette et al. Matrix biology: journal of the International Society for Matrix Biology, 2011; 30(5-6), 338-345. doi:10.1016/j.matbio.2011.05.001). Since the A allele of rs3196378 within the Hsa-miR-608 binding site was identified within the T functional form of the COL5A1 3′-UTR which was predominately cloned from TEN participants (Laguette et al. Matrix biology: journal of the International Society for Matrix Biology, 2011:30(5-6), 338-345. doi:10.1016/j.matbio.2011.05.001), we investigated the combined genotype frequencies MIR608 SNP rs4919510 and SNP rs3196378 (C/A, AciI RFLP) within the miRNA binding site. Although the M/R608CC and COL5A1 rs3196378 AA genotypes distributions were similar between the AUS TEN and AUS CON groups, the combined MIR608 CC genotype and COL5A1 rs3196378 C allele (CA and AA genotypes) were significantly over-represented in all the TEN participants when compared to all the CON participants.

The binding energy between the C allele of the mature miRNA and the A allele of its binding site was calculated to be the second most favourable. The most favourable was between the C alleles of both the Hsa-miR-608 and its binding sites. These calculations are calculated in silico and do not necessarily mimic the in vivo situation. The C form of Hsa-miR-608 bound the A rather than the C nucleotide of the SNP with higher affinity resulting in a corresponding decreased mRNA stability of the T allele.

The CC genotype of SNP rs12722 (BstUI RFLP), previously associated with chronic Achilles tendinopathy (Mokone et al. Scand J Med Sci Sports 2006; 16:19-26, September et al. Br J Sports Med 2009; 43:357-365), was over-represented in the participants with the genotypes of variants rs71746744 (2/1 AGGG and 1/1 AGGG), rs16399 (2/1 ATCT and 2/2 ATCT) and rs1134170 (TA and AA) not associated with Achilles tendinoapthy (FIGS. 8-10). This linkage of SNP rs12722, with rs71746744, rs16399 and rs1134170 is the simplest explanation for the previously reported associations. The association of the these three variants needs to be investigated within ACL ruptures (Posthumus et al. Am J Sports Med 37(11), 2009; 2234-2240) and other exercise-related phenotypes (Collins et al. Scand J Med Sci Sports 2009, 19(6), 803-810; Brown et al. Scand J Med Sci Sports, 2011 21(6), e266-72.) including ROM, athletic performance (Posthumus et al. Med Sci Sports Exerc 2011, 43(4), 584-589) and EAMC. The inventors have found that the COL5A1 SNP rs12722 is associated with endurance performance (FIG. 20) and (FIG. 21). Further work also has to be done to determine where SNP rs12722 (BstUI RFLP) has a direct effect on type V collagen production.

The third main finding of this study was the clear structural differences in the most stable C and T functional forms of the COL5A1 3′-UTR (refer to FIGS. 14 and 15). The predicted secondary structure of the region which contains both miRNA binding sites were distinctly different. Sequence differences within only seven polymorphic sites which span the entire 2.5 kb COL5A1 3′-UTR determined the distinct predicted secondary structures of the C and T functional forms. All seven of these polymorphic sites were to a less or greater extend responsible for determining the predicted structures associated with the C and T functional forms.

The data presented herein demonstrate that the MIR608 polymorphism investigated in this study interacts with the COL5A1 polymorphisms described herein in modifying the risk of AT. Although AT is likely to be a complex condition involving a number of gene-gene and gene-environment interactions (September et al. Br J Sports Med. 2007; 41:241-246), there have, to the Applicant's knowledge, been no such reports of a gene-gene interaction that relates to increased risk of AT. Furthermore the CASP8 polymorphisms described herein were unexpectedly found to increase risk of AT.

The invention relates to the association of the interactions of (i) rs16399 (ATCT/-) VNTR within the COL5A1 3′-untranslated region and the rs143383 (T/C) SNP within GDF5 (FIG. 21); (ii) rs16399 (ATCT/-) VNTR within the COL5A1 3′-untranslated region and the rs3834129 (CTTACT/del) polymorphism within CASP8 (FIG. 22); (iii) rs16399 (ATCT/-) VNTR within the COL5A1 3′-untranslated region and the rs1800795 (G/C) polymorphism within IL6 (FIG. 23) and (iv) rs16399 (ATCT/-) VNTR within the COL5A1 3′-untranslated region and the rs1143627 (T/C) polymorphism within IL1B all with increased risk of developing tendon, ligament, or other soft tissue pathology or injury related to other exercise related phenotypes, but not limited to, including ROM, endurance running performance and EAMC, Similar results were also noted for the interactions between polymorphisms within the COL5A1 gene (rs71746744; rs1134170) with each of the following polymorphisms (i) rs4919510 within MIR608 gene (ii) rs1045485 CASP8 gene (iii) and (iv) rs16944 within the IL1B gene and an increased risk of developing tendon, ligament, or other soft tissue pathology or injury related to other exercise related phenotypes, but not limited to, including ROM, endurance running performance and EAMC,

In one embodiment, the invention provides a method of determining in a subject a predisposition to, or increased risk for, developing a tendon, ligament, or other soft tissue injury or pathology related to other exercise related phenotypes, but not limited to, including ROM, endurance running performance and EAMC, the method comprising the step of screening the subject for the presence of at least one polymorphism in the MIR608 gene which encodes a miRNA which binds to a recognition sequence within the 3′-UTR of the collagen V gene COL5A1; and at least one polymorphism the collagen V gene COL5A1, which polymorphism is a polymorphism which results in a modified, augmented, or mitigated interaction with one or more other genes selected from the group, when compared to a wild-type interaction and wherein the presence of the polymorphism is indicative of a predisposition to, or increased risk for, developing a musculoskeletal soft tissue injury in the subject.

In a preferred embodiment, the polymorphism of the COL5A1 gene is selected from the group including rs71746744 (-/AGGG), rs16399 (ATCT/-) and rs1134170 (A/T) within the 3′-untranslated region (UTR) of the alpha 1 chain of the COL5A1 gene; and the polymorphism of the MIR608 gene is rs4919510 (C/G).

In another embodiment, the invention provides a method of determining in a subject a predisposition to, or increased risk for, developing a tendon, ligament, or other soft tissue injury or pathology, the method comprising the step of screening the subject for the presence of at least one polymorphism in the CASP8 gene, which polymorphism is a polymorphism which results in a modified, augmented, or mitigated interaction with one or more other genes selected from the group, when compared to a wild-type interaction and wherein the presence of the polymorphism is indicative of a predisposition to, or increased risk for, developing a musculoskeletal soft tissue injury in the subject.

The tendon, ligament, or other soft tissue injury or pathology may be a pathology related to other exercise related phenotypes, such as ROM, endurance running performance and EAMC.

In a preferred embodiment, the polymorphism of the CASP8 gene may be rs1045485 (G/C, D302H) and rs3834129 (CTTACT/del).

In a further embodiment of the invention, there is provided a DNA-based polymorphic marker molecular marker for use in diagnosing a predisposition to, or increased risk for, developing tendon, ligament, or other soft tissue pathology or injury in a subject, the molecular marker comprising at least one isolated nucleic acid fragment derived from a COL5A1 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof and at least one isolated nucleic acid fragment derived from a MIR608 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof.

More particularly, the molecular marker is as polymorphism selected from the group comprising rs71746744, rs16399 and rs1134170 within the 3′-untranslated region (UTR) of the alpha 1 chain of the COL5A1 gene and rs4919510 of the MIR608 gene.

In another embodiment of the invention, there is provided a DNA-based polymorphic marker molecular marker for use in diagnosing a predisposition to, or increased risk for, developing tendon, ligament, or other soft tissue pathology or injury in a subject, the molecular marker comprising at least one isolated nucleic acid fragment derived from a CASP8 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof.

The tendon, ligament, or other soft tissue injury or pathology may be a pathology related to other exercise related phenotypes, such as ROM, endurance running performance and EAMC.

In one embodiment, the molecular marker is a polymorphic marker, preferably a polymorphism including SNP rs1045485 and rs3834129 of the CASP8 gene.

What is claimed is:

1. A method of determining in a subject a predisposition to, or increased risk for, developing a tendon, ligament, or other soft tissue injury or pathology, the method comprising the step of screening the subject for the presence of at least one polymorphism in at least one gene selected from the group comprising:
a) the collagen V gene COL5A1; wherein the COL5A1 gene is rs71746744, rs16399 and/or rs1134170 within the 3′-untranslated region (UTR) of the alpha 1 chain of the COL5A 1 gene;
b) the MIR60B gene which encodes a miRNA which binds to a recognition sequence within the 3′-UTR of the collagen V gene COL5A 1; and
c) the CASP8 gene; wherein the presence of the polymorphism is indicative of a predisposition to, or increased risk for, developing a musculoskeletal soft tissue injury in the subject.

2-43. (canceled)
